(19) United States
(12) Patent Application Publication
(10) Pub. No.: US 2026/0055437 A1
(43) Pub. Date: Feb. 26, 2026

(54) BLOOD CULTURE DETECTION SYSTEM AND DETECTION METHOD THEREFOR

(71) Applicants: CHENGDU SHEN MINDRAY MEDICAL ELECTRONICS TECHNOLOGY RESEARCH INSTITUTE CO., LTD., Chengdu (CN); SHENZHEN MINDRAY BIO-MEDICAL ELECTRONICS CO., LTD., Shenzhen (CN)

(72) Inventors: Yabin DONG, Chengdu (CN); Wenjun TONG, Chengdu (CN); Jiayu ZOU, Chengdu (CN); Zhibin CHENG, Chengdu (CN)

(73) Assignees: CHENGDU SHEN MINDRAY MEDICAL ELECTRONICS TECHNOLOGY RESEARCH INSTITUTE CO., LTD., Chengdu (CN); SHENZHEN MINDRAY BIO-MEDICAL ELECTRONICS CO., LTD., Shenzhen (CN)

(21) Appl. No.: 19/307,160

(22) Filed: Aug. 22, 2025

(30) Foreign Application Priority Data
Aug. 22, 2024 (CN) 202411165475.6
Aug. 18, 2025 (CN) 202511157303.9

(51) Int. Cl. C12Q 1/04; G16H 10/40

(52) U.S. Cl. CPC C12Q 1/04; G16H 10/40

(57) ABSTRACT

A blood culture detection system and a detection method therefor. A controller acquires and processes a detection signal of a blood sample to obtain first information, while acquires second information related to metabolism of blood cells in a culture substrate, and obtains third information related to growth of micro-organisms based on the first information and the second information. On one hand, the first information is judged by using a first preset condition, to detect normal-growing micro-organisms. On the other hand, the third information obtained based on the first information and the second information is judged by using a second preset condition, to judge whether there are slow-growing micro-organisms in the sample.

### CROSS-REFERENCE TO RELATED APPLICATIONS

This application claims priority to Chinese patent application No. 202411165475.6 filed on Aug. 22, 2024 and Chinese patent application No. 202511157303.9 filed on Aug. 18, 2025. The disclosures of the above-referenced applications are hereby incorporated by reference in their entirety.

### BACKGROUND

A blood culture detection system is a microbiological detection device configured to detect whether bacteria are present in a blood sample, which plays a very important role of detecting whether there is growth of the bacteria in a patient's blood to make a definite diagnosis. A blood culture detection mechanism may incubate and detect the blood sample during use thereof, for example, may obtain a microbial growth curve of the blood sample by incubating and detecting the blood sample, such that doctors may make diagnoses according to the microbial growth curve. At present, the blood culture detection mechanism may analyze the microbial growth curve and obtain a detect result (for example, it may automatically judge whether the sample is positive or negative).

A maximum culture duration may be set in the blood culture detection system. If a positive result is obtained through analyzing the microbial growth curve before the maximum culture duration is reached, a positive report is outputted. If a positive result is not detected when the maximum culture duration is reached, a negative report is outputted. However, for some slow-growing micro-organisms, since they need special nutrients for their growth, culture flasks cannot meet requirements of their growth well, which may result in slow growth of the micro-organisms themselves, and then when the maximum culture duration is reached, the positive result still cannot be detected through the microbial growth curve, resulting in a false-negative result, which has a great impact on subsequent treatment of the patient.

### SUMMARY

The disclosure relates to the technical field of medical devices, and in particular to a blood culture detection system and a detection method therefor.

An overview of the subject matter described in detail in the disclosure is as follows. This overview is not intended to limit the scope of protection of the claims.

Embodiments of the disclosure provide a blood culture detection system and a detection method therefor, which can effectively improve a capability of detecting slow-growing micro-organisms and reduce false-negative results.

According to a first aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information;
if the first information meets a first preset condition within a first preset time period, label and output a detection status of the blood sample as “positive”;
if the third information meets a second preset condition within a first preset time period, label and output a detection status of the blood sample as “positive”.

In some embodiments of the disclosure, the controller acquires and processes the detection signal of the blood sample to obtain the first information, while acquires the second information related to metabolism of the blood cells in the culture substrate, and obtains the third information related to growth of the micro-organisms based on the first information and the second information. On one hand, the first information is judged according to the first preset condition, to detect normal-growing micro-organisms. On the other hand, the third information obtained based on the first information and the second information is judged according to the second preset condition, to judge whether there are slow-growing micro-organisms in the sample, which can effectively solve the false-negative problem in some implementations caused by the slow-growing micro-organisms cannot being detected as positive within the maximum culture duration due to their slow growth, significantly improve accuracy of the blood culture detection system, and reduce false-negative results.

According to a second aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container; and
obtain third information related to growth of the micro-organisms within the blood sample in the culture substrate of the sample container, based on the first information and the second information.

In some embodiments of the disclosure, the controller acquires and processes the detection signal of the blood sample to obtain the first information, while acquires the second information related to metabolism of the blood cells in the culture substrate, and obtains the third information related to growth of the micro-organisms based on the first information and the second information, such that growth states of the micro-organisms after removing the influence of metabolism of the blood cells can be obtained and observed according to the third information, to provide a more reliable basis for doctors' diagnoses and analysis of the blood culture detection system.

According to a third aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information; and
send a prompt of extending culture to a user or automatically extend culture, according to the third information.

In some embodiments of the disclosure, the controller acquires and processes the detection signal of the blood sample to obtain the first information, while acquires the second information related to metabolism of the blood cells in the culture substrate, obtains the third information related to growth of the micro-organisms based on the first information and the second information, and sends the prompt of extending culture to the user or automatically extends the culture according to the third information. Whether it needs to extend culture time is intelligently judged through analyzing growth states of the micro-organisms after removing the influence of metabolism of the blood cells, which can effectively solve the false-negative problem in some implementations caused by the slow-growing micro-organisms cannot being detected as positive within the maximum culture duration due to their slow growth, avoid a false-negative detection result of the slow-growing micro-organisms due to early termination of culture that may be caused by a traditional fixed culture time, significantly improve a detection rate of the slow-growing micro-organisms by the blood culture detection system, and provide a more accurate and reliable detection result for clinical diagnosis.

According to a fourth aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
if the first information meets a first preset condition within a first preset time period, label and output a detection status of the blood sample as “positive”, here the first preset condition is set based on an influence of metabolism of micro-organisms within the blood sample in the culture substrate on the detection signal of the blood sample; if the first information meets a fourth preset condition within a first preset time period, label and output an detection status of the blood sample as “positive”, here the fourth preset condition is set based on an influence of metabolism of blood cells within the blood sample in the culture substrate on the detection signal of the blood sample; or
if a culture time reaches an end time of the first preset time period, and the first information still does not meet the first preset condition while meets the fourth preset condition, send a prompt of extending culture to a user or automatically extend culture; and/or
if a culture time reaches an end time of the first preset time period, and the first information still does not meet the first preset condition and does not meet the fourth preset condition, label and output an detection status of the blood sample as “negative”, here the fourth preset condition is set based on an influence of metabolism of blood cells within the blood sample in the culture substrate on the detection signal of the blood sample.

In some embodiments of the disclosure, the controller acquires and processes the detection signal of the blood sample to obtain the first information, and judges the first information according to the first preset condition to detect normal-growing micro-organisms, and further judges the first information according to the fourth preset condition which is set based on the influence of metabolism of blood cells within the blood sample in the culture substrate on the detection signal of the blood sample, to detect slow-growing micro-organisms, which can effectively solve the false-negative problem in some implementations caused by the slow-growing micro-organisms cannot being detected as positive within the maximum culture duration due to their slow growth, significantly improve accuracy of the blood culture detection system, and reduce false-negative results.

In some embodiments of the disclosure, the first information is judged according to the first preset condition, to detect the normal-growing micro-organisms and label them as “positive” in time. In another embodiment, when a culture time reaches an end time of the first preset time period, the first information is judged according to the fourth preset condition; if the fourth preset condition is met, the prompt of extending culture is sent to the user or the culture is automatically extended; if the fourth preset condition is not met, the micro-organisms are labeled as “negative”. Early termination of culture that may be caused by a traditional fixed culture time is avoided by an intelligent extension culture mechanism, which can effectively solve the false-negative problem in some implementations caused by the slow-growing micro-organisms cannot being detected as positive within the maximum culture duration due to their slow growth, significantly improve a detection rate of the slow-growing micro-organisms by the blood culture detection system, reduce false-negative results, and provide a more accurate and reliable detection result for clinical diagnosis.

According to a fifth aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
if the first information meets a preset model within a first preset time period, label and output a detection status of the blood sample as “positive”; or
if a culture time reaches an end time of the first preset time period, and the first information does not meet the preset model, label the detection status of the blood sample as “negative” or “negative so far”; or
if the first information within the first preset time period conforms to a preset model, send a prompt of extending culture to a user or automatically extend culture, here the preset model is a model established based on an influence of metabolism of blood cells within the blood sample in the culture substrate on the detection signal of the blood sample.

In some embodiments of the disclosure, the controller acquires and processes the detection signal of the blood sample to obtain the first information, and judges the first information by using the preset model which is established based on the influence of metabolism of the blood cells within the blood sample in the culture substrate on the detection signal of the blood sample. When the first information meets the preset model, the detection status is labeled and outputted as “positive”, and when a culture time reaches an end time of the first preset time period, and the first information does not meet the preset model, the detection status is labeled as “negative” or “negative so far”, which can effectively solve the false-negative problem in some implementations caused by the slow-growing micro-organisms cannot being detected as positive within the maximum culture duration due to their slow growth, significantly improve accuracy of identifying different types of micro-organisms by the blood culture detection system through the preset model in which the influence of metabolism of the blood cells on the detection signal is considered, reduce false-negative results, and provide a more accurate and reliable detection result for clinical diagnosis.

In some embodiments of the disclosure, the first information is judged by using the preset model that is established based on the influence of metabolism of the blood cells within the blood sample in the culture substrate on the detection signal of the blood sample, and when the first information within the first preset time period conforms to the preset model, the prompt of extending culture is sent to the user or the culture is automatically extended. Requirements of extending the culture are intelligently judged to avoid the problem that the slow-growing micro-organisms cannot be identified due to early termination of culture that may be caused by a traditional fixed culture time, which can significantly improve a detection rate of the slow-growing micro-organisms by the blood culture detection system, reduce false-negative results, and provide a more accurate and reliable detection result for clinical diagnosis.

According to a sixth aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information;
if the first information does not meet a first preset condition within a first preset time period, and the third information does not meet a second preset condition within the first preset time period, label and output the detection status of the blood sample as “negative” or “negative so far”.

In some embodiments of the disclosure, the controller acquires and processes the detection signal of the blood sample to obtain the first information, acquires the second information related to metabolism of the blood cells in the culture substrate, and obtains the third information related to growth of the micro-organisms based on the first information and the second information. On one hand, the first information is judged according to the first preset condition, to detect normal-growing micro-organisms. On the other hand, the third information obtained based on the first information and the second information is judged according to the second preset condition. When the first information does not meet the first preset condition and the third information does not meet the second preset condition, the detection status is labeled and outputted as “negative” or “negative so far”, which can improve accuracy of the negative status by removing the influence of metabolism of the blood cells, and provide a more accurate and credible detection result for clinical diagnosis.

According to a seventh aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information; and
if the third information conforms to a second preset condition, label and output a detection status of the blood sample as “positive”; or, if the third information does not conform to the second preset condition, label the detection status of the blood sample as “negative so far” or “negative”.

In some embodiments of the disclosure, the controller acquires and processes the detection signal of the blood sample to obtain the first information, acquires the second information related to metabolism of the blood cells in the culture substrate, obtains the third information related to growth of the micro-organisms based on the first information and the second information, and judges the third information obtained based on the first information and the second information according to the second preset condition. In an embodiment, if the third information conforms to the second preset condition, the detection status is labeled and outputted as “positive”; in another embodiment, if the third information does not conform to the second preset condition, the detection status is labeled as “negative so far” or “negative”. The micro-organisms are detected and judged based on the third information after removing the influence of metabolism of the blood cells, which can effectively solve the false-negative problem in some implementations caused by the slow-growing micro-organisms cannot being detected as positive within the maximum culture duration due to their slow growth, significantly improve accuracy of detecting the micro-organisms by the blood culture detection system, reduce false-negative results, and provide a more accurate and credible detection result for clinical diagnosis.

According to an eighth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

If the first information meets a first preset condition within a first preset time period, a detection status of the blood sample is labeled and outputted as “positive”.

If the third information meets a second preset condition within the first preset time period, the detection status of the blood sample is labeled and outputted as “positive”.

According to a ninth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

The display is controlled to display the third information.

According to a tenth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

A prompt of extending culture is sent to a user or the culture is automatically extended, according to the third information.

According to an eleventh aspect, an embodiment of the disclosure provides a sample detection method, the sample detection method includes the following operations.

A detection module acquires a detection signal of a blood sample, and processes the detection signal to obtain first information of the blood sample.

If the first information meets a first preset condition within a first preset time period, a detection status of the blood sample is labeled and outputted as “positive”, here the first preset condition is set based on an influence of metabolism of micro-organisms within the blood sample in a culture substrate on the detection signal of the blood sample; if the first information meets a fourth preset condition within the first preset time period, the detection status of the blood sample is labeled and outputted as “positive”, here the fourth preset condition is set based on an influence of metabolism of blood cells within the blood sample in the culture substrate on the detection signal of the blood sample.

Or, if a culture time reaches an end time of the first preset time period, and the first information still does not meet the first preset condition while meets the fourth preset condition, a prompt of extending culture is sent to a user or the culture is automatically extended.

And/or, if an culture time reaches an end time of the first preset time period, and the first information still does not meet the first preset condition and does not meet the fourth preset condition, the detection status of the blood sample is labeled and outputted as “negative”, here the fourth preset condition is set based on an influence of metabolism of blood cells within the blood sample in the culture substrate on the detection signal of the blood sample.

According to a twelfth aspect, an embodiment of the disclosure provides a sample detection method, the sample detection method includes the following operations.

A detection module acquires a detection signal of a blood sample, and processes the detection signal to obtain first information of the blood sample.

If the first information meets a preset model within a first preset time period, a detection status of the blood sample is labeled and outputted as “positive”.

Or, if a culture time reaches an end time of the first preset time period, and the first information does not meet the preset model, the detection status of the blood sample is labeled as “negative” or “negative so far”.

Or, if the first information within the first preset time period conforms to a preset model, a prompt of extending culture is sent to a user or the culture is automatically extended, here the preset model is a model established based on an influence of metabolism of blood cells within the blood sample in a culture substrate on the detection signal of the blood sample.

According to a thirteenth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

If the first information does not meet a first preset condition within a first preset time period, and the third information does not meet a second preset condition within the first preset time period, the detection status of the blood sample is labeled and outputted as “negative” or “negative so far”.

According to a fourteenth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

If the third information conforms to a second preset condition, a detection status of the blood sample is labeled and outputted as “positive”; or, if the third information does not conform to the second preset condition, the detection status of the blood sample is labeled as “negative so far” or “negative”.

According to a fifteenth aspect, an embodiment of the disclosure provides an electronic device, the electronic device includes a memory and a control component, the memory stores a computer program, and the sample detection method according to any one of the above embodiments is implemented when the control component executes the computer program.

According to a sixteenth aspect, an embodiment of the disclosure provides a computer storage medium, the computer storage medium has stored thereon a computer program. The computer storage medium is applied to a pipeline system, and the sample detection method according to any one of the above embodiments is implemented when the computer program is executed by a control component.

According to a seventeenth aspect, an embodiment of the disclosure provides a computer program product or a computer program, the computer program product or the computer program includes computer instructions stored in a computer-readable storage medium. A control component of a computer device reads the computer instructions from the computer-readable storage medium, and the control component executes the computer instructions, such that the computer device executes the sample detection method according to any one of the above embodiments.

Other features and advantages of the embodiments of the disclosure will be set forth in the following description, part of which will become apparent from the description or will be understood by implementing the embodiments of the disclosure. Objects and other advantages of the embodiments of the disclosure may be achieved and obtained by structures particularly pointed out in the description, claims and drawings.

### BRIEF DESCRIPTION OF THE DRAWINGS

The drawings are intended to provide further understanding of technical solutions of the disclosure, constitute a part of the description, explain the technical solutions of the disclosure together with the embodiments of the disclosure, and do not constitute limitation on the technical solutions of the disclosure.

FIG. 1 is a schematic diagram of a reflectivity curve according to an embodiment of the disclosure.

FIG. 2 is a schematic diagram of a reflectivity curve according to another embodiment of the disclosure.

FIG. 3 is a block diagram of a system principle of a sample culture system according to an embodiment of the disclosure.

FIG. 4 is a flowchart of an operation principle of a sample culture system according to an embodiment of the disclosure.

FIG. 5 is a schematic diagram of a reflectivity curve according to another embodiment of the disclosure.

FIG. 6 is a flowchart of an operation principle of a sample culture system according to an embodiment of the disclosure.

FIG. 7 is a schematic diagram of an interface of extending culture according to an embodiment of the disclosure.

### DETAILED DESCRIPTION

The disclosure will be further described below with reference to the drawings and specific embodiments. The described embodiments should not be considered as limitation on the disclosure, and all other embodiments obtained by those of ordinary skill in the art without paying any creative work should fall within the scope of protection of the disclosure.

In the following descriptions, reference is made to “some embodiments” which describes a subset of all possible embodiments; however, it may be understood that “some embodiments” may be the same or different subsets of all possible embodiments, and may be combined with each other without conflict.

Unless otherwise defined, all technical and scientific terms used here have the same meaning as usually understood by technicians in the technical field to which the disclosure belongs. The terms used here are only for the purpose of describing the embodiments of the disclosure, and are not intended to limit the disclosure.

When a human body suffers from bloodstream infection (BSI)-related diseases such as bacteremia, fungemia or the like, it needs to collect blood from the patient and perform blood culture as soon as possible. At present, a gold standard for diagnosing the BSI is still the blood culture. Since treatment and prognosis of the patient suffered from the BSI infection are closely related to how quickly the diagnosis is made, while the blood culture is a first step of whether the infection is positive or negative, the time required to issue a report of the detection is critical for treatment of the patient. A blood culture detection mechanism may incubate and detect a blood sample during use thereof, for example, may obtain a microbial growth curve of the blood sample by incubating and detecting the blood sample, such that doctors may make diagnoses according to the microbial growth curve. At present, the blood culture detection mechanism may analyze the microbial growth curve and obtain a detection result (for example, it may automatically judge whether the sample is positive or negative).

Judgment of positive samples of the blood culture may rely on identifying characteristics of a detection signal, such as speed of a curve/point-to-point variation/area variation of the curve/slope variation/variation of absolute value of reflectivity, etc. Basic principles of these identification methods are that there is an exponential growth period for growth of the micro-organisms, and in this process, the reflectivity may increase significantly/have an accelerated increasing trend, which shows an apparent characteristic “head-up” on a reflectivity curve. A maximum culture duration during detection is set. If a positive result is obtained through analyzing the microbial growth curve before the maximum culture duration is reached, a positive report is outputted. If the positive result is not detected when the maximum culture duration is reached, a negative report is outputted. However, for some slow-growing micro-organisms, since they need special nutrients for their growth, culture flasks cannot meet requirements of their growth well, which may result in slow growth of the micro-organisms themselves, and when the maximum culture during is reached, the positive result still cannot be detected through the microbial growth curve, resulting in a false-negative detection result, which has a great impact on subsequent treatment of the patient.

Since one of samples collected for the blood culture is blood, the blood contains a large number of blood cells, metabolism of the blood cells produce acidic substances such as carbon dioxide or the like, and metabolism of bacteria also produce acidic substances such as carbon dioxide or the like, all of which are reasons for variations of signals of sensors at bottoms of the flasks. Therefore, metabolism of the blood cells may act as an interference factor which affect results of instruments' interpretation of the bacterial growth. When there are pronounced metabolism of the bacteria in the culture flasks, interference caused by metabolism of the blood cells is comparatively minor, which does not prevent a positive result from being reported. For example, with reference to FIG. 1, a curve A is a curve of a detection signal detected by a detection module, a curve B is a microbial growth curve in the detection signal, a curve C is a metabolism curve, and the curve A may be obtained by superimposing the curve B with the curve C. Since there are a fast growth speed and a large variation range in the microbial growth curve B of FIG. 1, the curve A of FIG. 1 may trigger positive reporting at time T3.

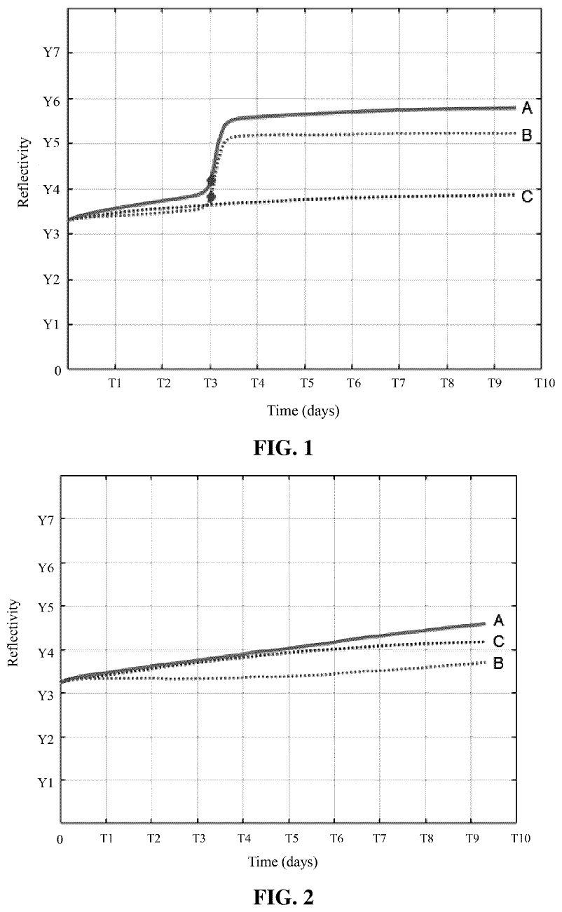

However, some micro-organisms themselves grow slowly, such as biphasic fungi, filamentous fungi, mycobacteria, etc. Since some bacteria need special nutrients for their growth, culture flasks cannot meet their growth well, such as some fastidious bacteria: Bartonella, Listeria genera, Brucella genera and Nocardia genera. When these micro-organisms are metabolized slowly in the culture flasks, interference caused by metabolism of the blood cells is pronounced, which may have a significant impact on whether a positive result can be reported accurately and the time of reporting the positive result. For slow-growing micro-organisms, since the micro-organisms grow slowly before the maximum culture duration (a minimum culture time required to clinically label and output a detection status of the blood sample as “negative”) is reached, and speed of a curve/point-to-point variation/area variation of the curve/slope variation/absolute value of reflectivity is small, resulting in that interference caused by metabolism of the blood cells is great, then it is difficult to observe an influence of microbial growth on shape of the curve before the maximum culture duration is reached. For example, with reference to FIG. 2, a curve A is a detection signal detected by a detection module, a curve B is a microbial growth curve in the detection signal, a curve C is a cell metabolic signal curve, and the curve A may be obtained by superimposing the curve B with the curve C. Since there is a slow growth in the microbial growth curve B of FIG. 2, it is unclear whether increase of numerical values of the detection signal curve A is caused by the microbial growth or the cell metabolic signal by simply analyzing the detection signal curve A, and then it is impossible to judge whether there are micro-organisms in the sample according to the detection signal curve A of FIG. 2.

Algorithms of existing solutions themselves cannot distinguish between blood metabolism characteristics and bacterial growth characteristics, and they need to compromise between making an accurate positive reporting and avoiding a false positive reporting, and therefore specificity and sensitivity of the algorithms are not high. When there are micro-organisms in the culture flasks and the micro-organisms grow slowly in the culture flasks, the instrument reports a positive result late-extending the time-to-positivity and delaying appropriate treatment of patients, even when the maximum culture duration is reached, the instrument still cannot report a positive result, which easily results in a false-negative detection result of slow-growing bacteria and has a great impact on subsequent treatment of the patient.

According to a first aspect, embodiments of the disclosure provide a blood culture detection system and a detection method therefor, which may effectively improve an ability of detecting slow-growing micro-organisms and reduce false-negative results.

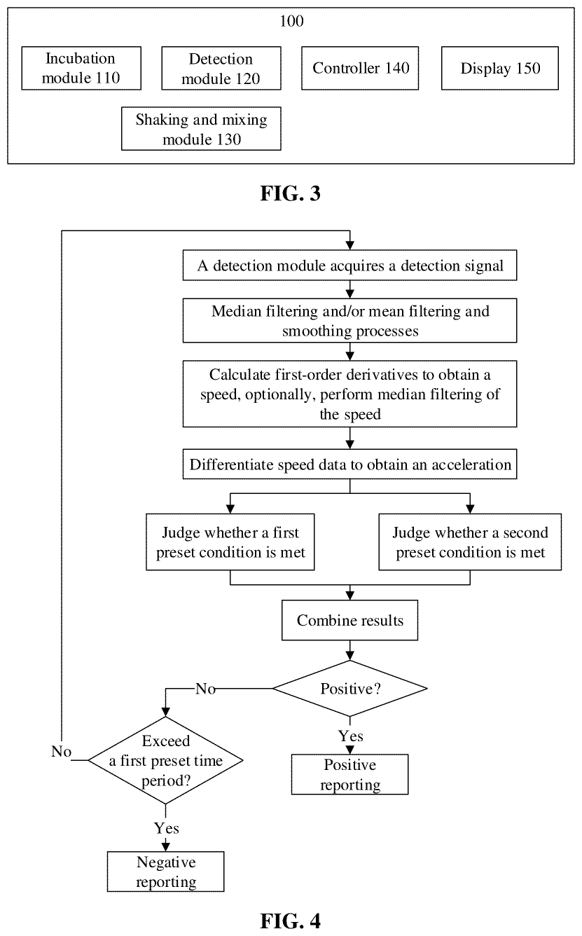

With reference to FIG. 3, a blood culture detection system 100 according to an aspect of the embodiments of the disclosure is shown, the blood culture detection system 100 includes an incubation module 110, a detection module 120, a shaking and mixing module 130, a controller 140 and a display 150. The detection module 120 is configured to detect samples held in the incubation module 110, the controller 140 is connected to the incubation module 110, the detection module 120 and the shaking and mixing module 130, the connection may include wired or wireless communication connection. In an embodiment, the incubation module 110 holds at least one sample and cultures micro-organisms within a blood sample, and the detection module 120 detects the blood sample and obtains a detection signal. The shaking and mixing module 130 is arranged in the incubation module 110 to shake and mix the blood sample before and/or during culture of the blood sample.

In an embodiment, the incubation module 110, the detection module 120, the shaking and mixing module 130, the controller 140 and the display 150 may be arranged in a shell component to form the blood culture detection system 100. In another embodiment, the incubation module 110, the detection module 120, the shaking and mixing module 130, the controller 140 and the display 150 may be arranged in a distributed manner, for example, they are communicatively connected through a bus or a local area network/Internet; or, a part of them form a sample culture detection device, and another part of them are external components connected to the sample culture detection device. For example, the incubation module 110, the detection module 120 and the shaking and mixing module 130 form a microorganism detection device arranged in a shell component, the controller 140 and the display 150 are externally connected to the microorganism detection device to form the blood culture detection system 100. Of course, different combinations of components may also be provided, which is not limited in the disclosure.

It should be noted that the shaking and mixing module 130 in the above embodiment is not essential, and the shaking and mixing module 130 may not be provided in some embodiments.

The detection module 120 includes at least one detection sensor configured to detect the blood sample and to generate a detection signal of the blood sample.

The detection sensor may detect the blood sample in different detection manners. For example, in an embodiment, the detection sensor is an optical sensor configured to detect changes of a reflectivity of a chromogenic film at the bottom of a sample container, and the optical sensor includes one or more light sources (such as Light Emitting Diodes (LEDs), laser diodes, etc.) and one or more photodetectors (such as photodiodes, phototransistors, etc.). The light source emits a light signal to the chromogenic film at the bottom of the sample container, and the chromogenic film generates a chromogenic reaction with acidic substances produced during culture. With the growth and reproductions of the micro-organisms, the produced acidic substances increase, and color of the chromogenic film changes, thereby changing reflection characteristics of the chromogenic film to light. The photodetector detects changes of intensity of the reflected light signal to obtain a detection signal that represents how reflectivity varies over culture time, and the controller may process the detection signal to obtain a curve of the reflectivity versus the culture time.

In another embodiment, the detection sensor is a fluorescence sensor configured to detect changes of fluorescence intensity of a fluorescent substance in the sample container, the sample container contains a specific fluorescent substance of which fluorescence intensity changes with growth of micro-organisms. With the growth and reproductions of the micro-organisms, the fluorescence intensity changes correspondingly, and the detection module 120 obtains a detection signal that represents how the fluorescence intensity varies over culture time through the fluorescence sensor, and the controller may process the detection signal to obtain a curve of the reflectivity versus the culture time.

In another embodiment, the detection sensor is a pressure sensor configured to detect changes of pressure in a head space of the sample container, here the changes of pressure is caused by gases generated and/or consumed by metabolism of blood cells within the blood sample in a culture substrate of the sample container and growth of micro-organisms within the blood sample in the culture substrate of the sample container, the pressure sensor monitors the changes of pressure within the container. With the growth and reproductions of the micro-organisms, the generated or consumed gases increase, the changes of pressure is more pronounced. The detection module 120 obtains a detection signal that represents how the gas pressure varies over culture time through the pressure sensor, and the controller may process the detection signal to obtain a curve of the reflectivity versus the culture time.

It should be noted that no matter which detection manner is used, the detection signal may be stored in an array manner in addition to being presented in a curve manner, for example, numerical values of the reflectivity, the fluorescence intensity or the gas pressure corresponding to different culture times. After these detection signals are processed, the curve of the reflectivity versus the culture time, the curve of the fluorescence intensity versus the culture time and the curve of the gas pressure versus the culture time may be obtained respectively, which provides data bases for subsequent detection and judgment of the micro-organisms.

The controller 140 and the detection module 120 obtain the detection signal in any one of the above embodiments, and process the detection signal to obtain first information of the blood sample. In an embodiment, the controller 140 may control the display 150 to display the detection signal or corrected information (for example, to display a growth curve of the slow-growing micro-organisms), and the display 150 may provide a human-machine interaction interface through which a user may view the growth curve or perform operations such as labeling and calculating the growth curve, etc.

In an embodiment, after obtaining the detection signal, the controller 140 may also perform pre-processing on the obtained detection signal to obtain the first information. The pre-processing may be filtering and smoothing the detection signal for subsequent operations, the filtering and smoothing may use classical median filtering or mean filtering or a combination thereof.

In an embodiment, the controller 140 may analyze the first information automatically or with manual intervention, to judge whether there are micro-organisms in the blood sample through the first information, and if there are micro-organisms, the controller 140 may report a positive result. In an embodiment, if the micro-organisms are detected in a first preset time period in which the blood sample is first cultured to a minimum culture time required to clinically label and output a detection status of the blood sample as “negative”, the detection status of the blood sample may be labeled and outputted as “positive”, for example, as long as the micro-organisms are detected according to the first information, the detection status of the blood sample may be labeled as “positive”, and a positive report (also referred to as positive reporting) may be output. In an embodiment, positive reporting may be made immediately when the micro-organisms are detected, or positive reporting may be made according to a preset reporting time, for example, the detection status of the blood sample may be determined every one hour, and if it is found that the detection status of the blood sample is positive, positive reporting may be made. If the micro-organisms are not detected in the first preset time period in which the blood sample is first cultured to the minimum culture time required to clinically label and output the detection status of the blood sample as “negative”, the detection status of the blood sample may be labeled and outputted as “negative so far”, and if the detection status of the blood sample is still “negative so far” beyond the above first preset time period, a negative report (also referred to as negative reporting) may be output; or, negative reporting may not be made according to some conditions (such as a third preset condition mentioned below), and a prompt of extending culture is sent to the user, or the culture time of the blood sample is automatically extended. In an embodiment, as long as the blood sample is arranged in the incubation module 110 and is not taken out (i.e., unloaded), the detection module 120 may continuously detect the blood sample even when an end time of the first preset time period is reached. Therefore, the prompt of extending culture may be sent to the user, to inform the user not to take out (i.e., unload) the blood sample. In this way, an effect of automatically extending the culture time may also be achieved.

In an embodiment, with reference to FIG. 4, the controller 140 determines whether the detection signal conforms to a first preset condition and a second preset condition, the first preset condition is set for growth of ordinary micro-organisms, micro-organisms with apparent metabolism may be found according to the first preset condition, and the second preset condition is set for slow-growing micro-organisms with slow growth speeds. As mentioned above, the growth curve of the slow-growing micro-organisms is affected by metabolism of the blood cells within the blood sample, and the blood cells within the blood sample form cell metabolic signals that may be detected by the detection module 120 due to metabolism. Therefore, the second preset condition considers an influence of a cell metabolic signal model on the detection signal, that is, the second preset condition is set based on the influence of the cell metabolic signal model on the detection signal.

On one hand, if the first information meets a first preset condition within a first preset time period, a detection status of the blood sample is labeled and outputted as “positive”.

On the other hand, the controller 140 may acquire second information related to metabolism of blood cells within the blood sample in the culture substrate of the sample container, and obtain third information related to growth of the micro-organisms within the blood sample in the culture substrate of the sample container, based on the first information and the second information. The third information is information in which relevant interference related to metabolism of the blood cells in the culture substrate of the sample container has been removed. If the third information meets a second preset condition within the first preset time period, there are slow-growing microorganism in the blood sample, and the controller 140 labels and outputs the detection status of the blood sample as “positive”.

That is, as long as at least one of the first preset condition or the second preset condition is met in the first preset time period, the detection status of the blood sample is labeled as “positive”. In an embodiment, the first preset time period is 5 days, 7 days or 14 days from an initial culture of the blood sample. In some embodiments, after the blood sample is held, the first information of the blood sample may be judged by using the first preset condition after a certain culture time. For example, a judgment algorithm of the first preset condition may be started after the culture time of the blood sample elapses by 2 hours. In some embodiments, an early algorithm may also be used, and the judgment algorithm of the first preset condition is started after the blood sample is cultured for 0.5 hours. Alternatively, in some embodiments, execution of the judgment algorithm of the first preset condition is started after entering the first preset time period.

In some embodiments of the disclosure, the controller 140 acquires and processes the detection signal of the blood sample to obtain the first information, simultaneously acquires the second information related to metabolism of the blood cells in the culture substrate, and obtains the third information related to growth of the micro-organisms based on the first information and the second information. On one hand, the first information is judged according to the first preset condition, to detect normal-growing micro-organisms. On the other hand, the third information obtained based on the first information and the second information is judged according to the second preset condition, to judge whether there are slow-growing micro-organisms in the sample. Therefore, it may effectively solve the false-negative problem in some implementations caused by the slow-growing micro-organisms cannot being detected as positive within the maximum culture duration due to their slow growth, significantly improve accuracy of the blood culture detection system, and reduce false-negative results.

In an embodiment, the controller 140 applies the first preset condition and the second preset condition to the first information generated from the detection signal acquired and processed by the detection module 120 and the third information obtained based on the first information and the second information respectively to perform judgments thereof, both the first preset condition and the second preset condition are threshold conditions, that is, the controller 140 judges whether the first information corresponding to the detection signal exceeds a threshold of the first preset condition, and judges whether the third information exceeds a threshold of the second preset condition. The threshold of the first preset condition is higher than the threshold of the second preset condition, and in some embodiments, the threshold of the first preset condition is higher than the threshold of the second preset condition by at least one order of magnitude (that is, higher by at least 10 times).

In an embodiment, in addition to processing the first information through the second information to obtain the third information, the controller 140 may perform regulation during processing according to a blood volume information of the blood sample. For example, the controller 140 may find or estimate the second information characterizing a corresponding cell metabolic signal from a preset blood volume-metabolic signal mapping table according to a blood volume of the blood sample, and may dynamically adjust a processing coefficient or weighting ratio when superimposition, subtraction or proportional operation is performed on the first information and the second information, such that the third information may reflect changes of signals generated by growth of micro-organisms in samples with different blood volumes more accurately. It should be noted that in other embodiments of the disclosure, any solution of processing the first information based on the second information may be adjusted based on the blood volume of the blood sample. For example, a processing degree is controlled by providing different processing coefficients, proportions or mapping parameters, thereby further improving the capability of detecting the slow-growing micro-organisms, and reducing false or missed results caused by variations in blood volume.

For example, obtaining the third information related to growth of the micro-organisms within the blood sample in the culture substrate of the sample container, based on the first information and the second information includes that: the third information is obtained by removing the second information from the first information; or, the third information may be obtained by removing the second information from the first information according to the blood volume of the blood sample. For example, the more the blood volume, the stronger the blood cell metabolic signal, then a weight of removing the second information may be increased when the second information is removed, and the weight of removing the second information may be reduced when the blood volume is small.

Different types of first information may be processed in different manners, and the first information is a detection curve generated based on detection signals of the blood sample at different culture times, that is, a detection curve. Correspondingly, the second information is a metabolism curve related to metabolism of the blood cells within the blood sample in the culture substrate of the sample container at different culture times, and the third information obtained by processing the detection curve according to the metabolism curve is a growth curve related to growth of the micro-organisms within the blood sample in the culture substrate of the sample container at different culture times.

In an embodiment, each of the detection curve, the metabolism curve and the growth curve is a curve of numerical values versus time (that is, a continuous representation manner), here the abscissa of the curve is incubation time, and numerical values of the ordinate are related to types of the detection sensor.

In an embodiment, the detection sensor is an optical sensor configured to detect changes of a reflectivity of a chromogenic film at the bottom of the sample container, and each of the detection curve, the metabolism curve and the growth curve is a curve of the reflectivity versus the culture time. In another embodiment, the detection sensor is a fluorescence sensor configured to detect changes of a fluorescence intensity of a fluorescent substance in the sample container, and each of the detection curve, the metabolism curve and the growth curve is a curve of the fluorescence intensity versus the culture time. In another embodiment, the detection sensor is a pressure sensor configured to detect changes of a pressure in a head space of the sample container, and each of the detection curve, the metabolism curve and the growth curve is a curve of the gas pressure versus the culture time.

It should be noted that the growth curve represents a curve reflecting how the number of micro-organisms changes over time during culture. For example, it may be judged through reflectivity when the micro-organisms enter a logarithmic growth phase (that is, “ramp-up”) and subsequent growth or stable states.

In an embodiment, a curve representing how detected numerical values (for example, reflectivity) changes over culture time may also be converted into a curve representing how the number of the micro-organisms changes over culture time.

In an embodiment, in addition to directly reflecting detected numerical values of the detection sensor, the curve may be a curve obtained by processing the detected numerical values, and for example, may be a speed curve (first-order derivatives of the curve of detected numerical values), an acceleration curve (second-order derivatives of the curve of detected numerical values), etc.

In an embodiment, the detection curve is processed according to the metabolism curve, to obtain the growth curve. In another embodiment, the metabolism curve is subtracted from the detection curve, to obtain the growth curve.

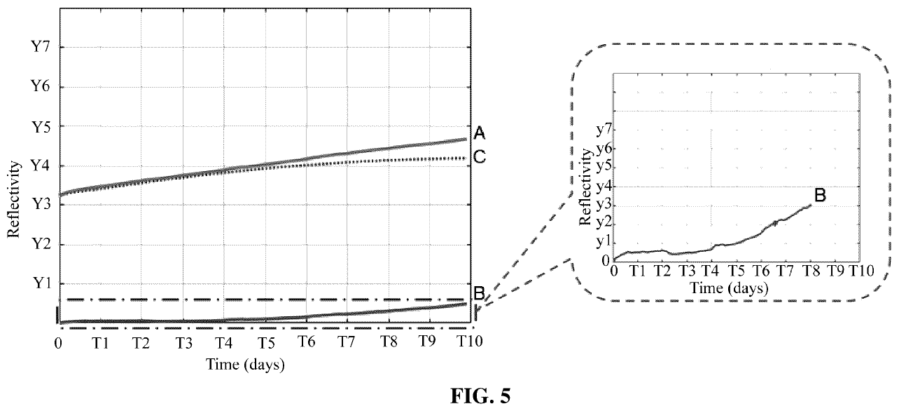

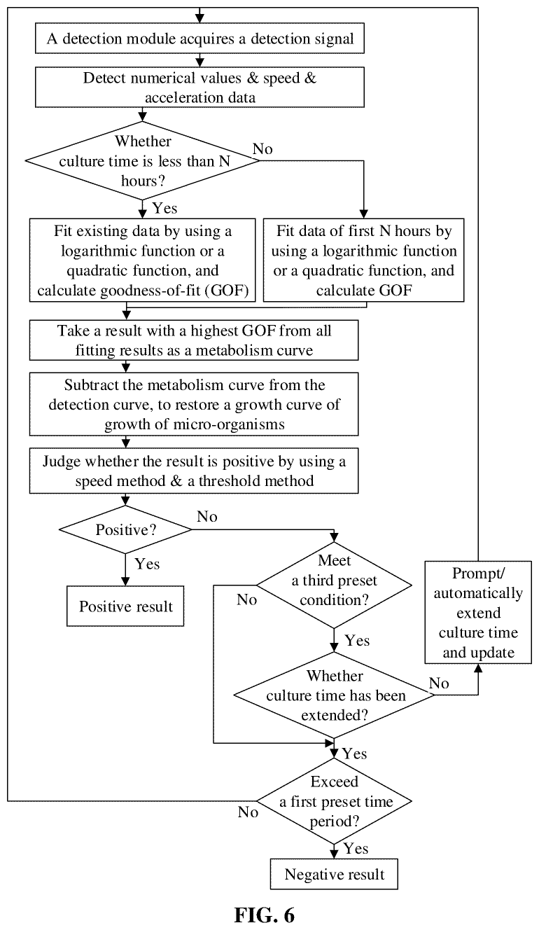

In another embodiment, the metabolism curve may also be determined according to the blood volume of the blood sample, and the metabolism curve is subtracted from the detection curve to obtain the growth curve, or weighted summation of the detection curve and the metabolism curve may be performed to obtain the growth curve, coefficients of the weighted summation may be determined according to the blood volume of the blood sample. For example, with reference to FIG. 5, a curve A is a detection curve obtained by processing a detection signal detected by a detection module, a curve B is a growth curve related to growth of the micro-organisms within the blood sample in the culture substrate of the sample container at different culture times, and a curve C is a metabolism curve related to metabolism of the blood cells within the blood sample in the culture substrate of the sample container at different culture times. In an embodiment, the target signal curve B is obtained by subtracting the curve C from the curve A. As may be know from an enlarged diagram of the curve B in FIG. 5, the growth curve B obtained by processing the detection curve A with the metabolism curve C may reflect slight changes of a growth trend of the micro-organisms.

In an embodiment, the first information is a detection curve generated based on detection signals of the blood sample at different culture times, and the above metabolism curve is obtained by performing a matching process based on the detection curve. Preferably, the metabolism curve is obtained by perform a similarity matching process based on the detection curve.

In another embodiment, the first information is discrete data obtained based on the detection signals of the blood sample at different culture times, and the above metabolism curve is obtained by fitting the discrete data.

In another embodiment, the first information may also be represented by discrete data obtained based on the detection signals of the blood sample at different culture times, for example, in an array or list manner, such that the second information may also be discrete data at different culture times. When processing the first information, subtraction or weighted summation may be performed on discrete data of the first information and the second information at corresponding culture times, to obtain the third information. Similarly, coefficients of the weighted summation may be determined according to the blood volume of the blood sample.

In an embodiment, when a first number of numerical values (which may be numerical values of discrete data or numerical values of data points in the growth curve) exceeding a first threshold continuously occur in the third information, it is determined that the second preset condition is met. For example, when continuous ml numerical values in the growth curve or discrete data of the third information exceed a set first threshold, it is determined that the second preset condition is met, and the detection status of the blood sample is labeled as “positive”, or positive reporting is made.

In another embodiment, for the third information, it may also be set that a time when the numerical value in the third information continuously exceeds the first threshold is not less than a first time interval threshold T1, then it is determined that the second preset condition is met, and the detection status of the blood sample is labeled as “positive”, or positive reporting is made.

Besides, speed values of variations of numerical values corresponding to different incubation times may also be calculated according to the third information, and when there continuously occurs a second number of first-order derivatives of the growth curve that are equal to or greater than a second threshold, it is determined that the second preset condition is met. In an embodiment, when a time interval in which first-order derivatives of the growth curve are continuously equal to or greater than a second threshold is equal to or longer than a second time interval threshold, it may also be determined that the second preset condition is met.

In an embodiment, acceleration values of variations of numerical values corresponding to different incubation times may also be calculated according to the third information, and when there continuously occurs a third number of second-order derivatives of the growth curve that are equal to or greater than a third threshold, it is determined that the second preset condition is met. In an embodiment, when a time interval in which second-order derivatives of the growth curve are continuously equal to or greater than a third threshold is equal to or longer than a third time interval threshold, it may also be determined that the second preset condition is met.

In an embodiment, the above multiple different calculation methods may also be performed simultaneously, and as long as one of the calculation methods conforms to the second preset condition, the detection status of the blood sample is labeled as “positive”, or positive reporting is made. When the numerical values of the growth curve continuously exceed the first threshold or the first-order derivatives of the growth curve continuously exceed the second threshold, it is determined that the second preset condition is met. That is, when a result of any one of the above calculation methods conforms to the second preset condition as mentioned above, positive reporting is made.

In an embodiment, the above speed values of variation of the numerical values (for example, the first-order derivatives of the growth curve) or the above acceleration values of variation of the numerical values (for example, the second-order derivatives of the growth curve) may be obtained by processing the detection signal, or may be obtained by directly calculating derivatives (first-order difference) of the growth curve to obtain a speed curve, and then comparing the speed curve to a corresponding threshold. Similarly, derivatives of the speed curve are calculated again (speed data is differentiated) to obtain an acceleration curve.

In the above embodiments, it is required that the third information continuously exceeds a corresponding threshold, and then it is determined that the second preset condition is met, to avoid a false reporting phenomenon caused by an individual numerical value(s) exceeding the threshold due to signal interference. Of course, a threshold may also be set, and as long as the numerical value of the third information is greater than the threshold, it is considered that the second preset condition is met, and it is unnecessary to count multiple pieces of data.

In an embodiment of the disclosure, for the first preset condition, it may also refer to the above manners of the second preset condition, for example, if the detection curve meets the first preset condition within the first preset time period, the detection status of the blood sample is labeled and outputted as “positive”.

The first preset condition includes at least one of the following conditions.

There continuously occurs a fourth number of numerical values of the detection curve that are equal to or greater than a fourth threshold, or a time interval in which numerical values of the detection curve are continuously equal to or greater than a fourth threshold is equal to or greater than a fourth time interval threshold.

Or, there continuously occurs a fifth number of first-order derivatives of the detection curve that are equal to or greater than a fifth threshold, or a time interval in which first-order derivatives of the detection curve are continuously equal to or greater than a fifth threshold is equal to or greater than a fifth time interval threshold.

Or, there continuously occurs a sixth number of second-order derivatives of the detection curve that are equal to or greater than a sixth threshold, or a time interval in which second-order derivatives of the detection curve are continuously equal to or greater thana sixth threshold is equal to or greater than a sixth time interval threshold.

Besides, in another embodiment, both the first-order derivatives and the second-order derivatives of the detection curve are equal to or greater than their corresponding preset thresholds either for a duration time interval that is equal to or greater than a preset time interval, or for a number of data points that is equal to or greater than a preset number.

That is, when both the speed values and the acceleration values are greater than their corresponding preset thresholds, either for a duration that is not less than a fourth time interval, or for a number of data points that is equal to or greater than the preset number, it is determined that the first preset condition is met. For example, when the speed values and the acceleration values of the detection curve are greater than their corresponding preset thresholds for the first time, timing is started or the number of data points is accumulated, and if a continuous time period in which both the speed values and the acceleration values are greater than respective preset thresholds is not less than a preset threshold, or the number of accumulated data points for which both the speed values and the acceleration values are greater than respective preset thresholds is greater than the preset number, it is proved that the micro-organisms in the blood sample has a strong growth trend, and it may be determined that the first information conforms to the first preset condition, that is, the first information conforms to a condition of making positive reporting.

Of course, judgment may be performed by merely using speed values of variations of detected numerical values in the first information. For example, speed values of variations of detected numerical values corresponding to different culture times are determined according to the detection curve, and when a fourth number of speed values exceeding the fourth threshold continuously occur, or a time duration in which the speed values continuously exceed the fourth threshold reaches the fourth time interval threshold, it is determined that the first preset condition is met. It may be seen that the same is true for judgment of the acceleration values, which will not be repeatedly elaborated here.

Furthermore, in an embodiment, positive detection may also be performed by determining numerical values of the first information in the first preset time period in which the blood sample is first held. For example, in the first information corresponding to the first preset time period, when a fifth number of numerical values exceeding the fifth threshold continuously occur, or a time duration in which the numerical values continuously exceed the fifth threshold reaches the fifth time interval threshold, it is determined that the first preset condition is met.

The first threshold, the second threshold, the third threshold, the fourth threshold and the fifth threshold in the above embodiments may be configured according to experiences or actual detection requirements, specific numerical values thereof will not be described here.

The first time interval threshold, the second time interval threshold, the third time interval threshold, the fourth time interval value and the fifth time interval threshold in the above embodiments may also be configured according to experiences or actual detection requirements, and may be set in basic units of days, hours, minutes and seconds.

In an embodiment, the speed values or acceleration values of variations of detected numerical values in the above first information may be obtained by performing calculation on the discrete data of the first information, or may be obtained by directly calculating derivatives of the detection curve to obtain a speed curve, and then comparing the speed curve with a corresponding threshold. Similarly, derivatives of the speed curve are calculated again to obtain an acceleration curve.

In an embodiment, a machine learning model trained in advance may be used to judge whether the third information conforms to the second preset condition. The controller 140 inputs the third information within the first preset time period to the machine learning model trained in advance, to perform judgment to confirm whether the third information conforms to the second preset condition. The machine learning model is obtained by training with a training set containing at least information related to metabolism of the blood cells within the blood sample in the culture substrate.

In another embodiment, a machine learning model trained in advance may also be used to judge whether the first information conforms to the first preset condition. The controller 140 inputs the first information within the first preset time period to a machine learning model trained in advance, to perform judgment to confirm whether the first information conforms to the first preset condition. The machine learning model is obtained by training with a training set containing at least information related to growth of the micro-organisms within the blood sample.

In an embodiment, detection opportunity for the above first preset condition may be the same as or different from detection opportunity for the above second preset condition. For example, the controller 140 acquires the first information within the first preset time period, and labels the detection status of the blood sample as “positive” if the first information within the first preset time period conforms to the first preset condition. Furthermore, the controller 140 processes a detection signal of the blood sample within a second preset time period to obtain the second information, and obtains the third information related to growth of the micro-organisms within the blood sample in the culture substrate of the sample container, based on the first information and the second information. In an embodiment, the detection signal of the blood sample within the second preset time period is substantially originated from metabolism of the blood cells within the blood sample in the culture substrate of the sample container. In an embodiment, the second preset time period may be set according to medical guidelines or requirements of clinical detection of the user, for example, the second preset time period may be set to be within 4 hours or 8 hours from an initial culture of the blood sample. In an embodiment, setting of the second preset time period is related to an elapsed time from blood collection time of the blood sample, and a time interval between the blood collection time of the blood sample and a time of loading the blood sample into the blood culture detection system is referred to as a delayed loading time. The longer the delayed loading time, the longer the growth time of the micro-organisms within the blood sample, and the second preset time period may be set to be shorter.

In another embodiment, the second preset time period may also coincide with the first preset time period, or it may be understood that the second preset time period is not set. When judging whether the first information conforms to the first preset condition, the second information may also be synchronously acquired and the third information may be generated, and acquisition of the first information, acquisition of the second information and generation of the third information are all performed within the first preset time period.

In an embodiment, the operation of processing the detection signal of the blood sample within the second preset time period to obtain the second information, includes the following contents. The detection signal of the blood sample within the second preset time period is processed to obtain a processing result, and the second information is generated based on the processing result. In another embodiment, the detection signal of the blood sample within the second preset time period is processed to obtain a processing result, and the second information is obtained by performing a matching process on the processing result from multiple pieces of preset information. In another embodiment, the second information is selected from multiple pieces of preset information based on a blood volume of the blood sample. The multiple pieces of preset information are configured to characterize metabolism of blood cells within multiple blood samples with different blood volumes in the culture substrate respectively.

If the third information within a third preset time period conforms to the second preset condition, the detection status of the blood sample is labeled as “positive”. In an embodiment, the first preset time period includes the second preset time period and is longer than the second preset time period. In an embodiment, the first preset time period includes the third preset time period, the second preset time period does not include the third preset time period but may partially coincide with the third preset time period, and a starting point of the second preset time period is earlier than a starting point of the third preset time period. In an embodiment, the presence of micro-organisms in the blood sample can be judged according to the first preset condition upon initial loading of the blood sample, whereas the second preset condition cannot be applied until the second information has been acquired and used to process the detection signal into the third information. In an embodiment, acquisition of the second information requires real-time analysis of the first information, or is achieved by acquiring and analyzing data in a certain time period, therefore the starting point of the third preset time period is later than a starting point of the first preset time period.

In another embodiment, the second preset time period may completely coincide with the third preset time period, or at least the starting point of the second preset time period is the same as the starting point of the third preset time period. For example, the second information is preset according to experiences (for example, the second information is calculated according to the blood volume information of the blood sample before starting detection or according to user configuration, or the second information is a preset default signal), such that the blood sample may be judged and detected by using both the first preset condition and the second preset condition upon initial loading of the blood sample, that is, the second preset time period completely coincides with the third preset time period. Alternatively, processing of the third information and judgment of the first preset condition and the second preset condition are actually performed in the first preset time period, without distinguishing the second preset time period and the third preset time period.

In an embodiment, the second information may be information generated according to the first information. For example, in an embodiment, the controller 140 generates the second information in real time based on the first information, for example, performs calculation on the whole data of current first information or data of the current first information in a specific culture time period according to a preset algorithm rule, to obtain the second information. Alternatively, a preset algorithm rule (for example, a fitting algorithm mentioned below) is adjusted based on the blood volume information of the blood sample, to performs calculation on the whole data of the current first information or data of the current first information in a specific culture time period, to obtain the second information.

In another embodiment, the second information is a piece of preset information, which may be preset default information, or may be determined based on the user's instructions or parameters of the blood sample. For example, the second information may be information selected by the user from multiple pieces of preset information, or information obtained by matching from the multiple pieces of preset information based on the first information (for example, the information is selected based on detected numerical values of the first information, the first-order derivatives of the detection curve or the second-order derivatives of the detection curve), or information obtained by matching from the multiple pieces of preset information according to the first information (for example, matching in a database of preset information by an approximation degree of the detection curve or characteristic parameters).

In an embodiment, the controller 140 is further configured to obtain the metabolism curve by performing a matching process based on the detection curve.

For example, in an embodiment, the metabolism curve is obtained by performing a matching process based on the detection curve within a second preset time period, a detection signal of the blood sample within the second preset time period is substantially originated from metabolism of the blood cells within the blood sample in the culture substrate of the sample container.

In another embodiment, the detection curve is periodically fitted during culture, to update the metabolism curve.

In another embodiment, within the second preset time period, the metabolism curve is periodically obtained by performing a matching process on the detection curve; outside the second preset time period, at least one metabolism curve obtained within the second preset time period is taken as a metabolism curve to be used currently; the detection signal of the blood sample within the second preset time period is substantially originated from metabolism of the blood cells within the blood sample in the culture substrate of the sample container. In an embodiment, the metabolism curve is periodically obtained by similarity matching based on the detection curve.

In an embodiment, discrete data is obtained by the detection signals of the blood sample at different culturing times, and the discrete data is fitted, to obtain the metabolism curve.

In an embodiment, with reference to FIG. 6, the above fitting operation includes the following contents.

The discrete data is fitted, to obtain metabolism curve, the second preset time period may be first N hours after the initial loading of the blood sample, for example, within 4 hours or 8 hours.

The discrete data is continuously fitted to obtain or update the metabolism curve.

If incubation time of the blood sample is within the second preset time period, the discrete data is continuously fitted to obtain or update the metabolism curve, until the incubation time of the blood sample exceeds the second preset time period, and then the discrete data within the second preset time period is fitted to obtain the metabolism curve.

After the initial loading of the blood sample, for slow-growing micro-organisms, the first information is mainly contributed by the second information in this time period due to hysteresis of their growth, and thus the metabolism curve may be predicted by fitting the discrete data within this time period. In order to improve real-time detection of the blood sample with the slow-growing micro-organisms, fitting the discrete data may be started after loading of the blood sample, the growth curve may be obtained according to the metabolism curve and the detection curve, and it may be judged whether the blood sample is positive according to the growth curve. Since the detection curve is continuously updated, a better fitting curve may be present during update, then fitting may be repeatedly performed and the metabolism curve is updated to process the detection curve, until positive reporting that conforms to the second preset condition is detected according to the growth curve or it exceeds the second preset time period. In an embodiment, considering that slow-growing micro-organisms (if any) in the blood sample may be reflected in the detection curve after exceeding the second preset time period, while a cell metabolism curve obtained by fitting the detection curve at this time is inaccurate, after exceeding the second preset time period, a latest fitting curve in the second preset time period or a fitting curve with a highest fitting degree is used as the metabolism curve used after the second preset time period. In an embodiment, the starting point of the second preset time period is earlier than the starting point of the third preset time period.

In another embodiment, fitting may not be limited as being performed in the second preset time period, the metabolism curve may also be updated continuously, or the metabolism curve may not be updated, instead, fitting may be performed once or a preset number of times according to the detection curve in a certain time period, and then the fitted curve may be used as a current metabolism curve. In another embodiment, in addition to determining the metabolism curve by fitting the detection curve, the discrete data of the first information may be compared and matched to determine a cell metabolic signal (for example, composed of discrete data points), that is, the discrete data is obtained based on the detection signals of the blood sample at different culture times, the metabolism curve is obtained by fitting the discrete data.

In an embodiment, the controller is further configured to obtain the metabolism curve by fitting the discrete data with a fitting function, the fitting function includes at least one of a logarithmic fitting function or a quadratic fitting function.

In an embodiment, the logarithmic fitting function is expressed by a following formula: y=A1*log_{B1}Xᵢ+C1, here Xᵢ is a culture time, y is a numerical value of the detection curve, A1, B1 and C1 are variable fitting parameters.

In an embodiment, the quadratic fitting function is expressed by a following formula: y=A2*Xᵢ²+B2*Xᵢ+C2, herein Xᵢ is a culture time, y is a numerical value of the detection curve, A2, B2 and C2 are variable fitting parameters.

The variable fitting parameters in the above embodiments may also be set according to adjustment parameters in the above embodiments. For example, the above variable fitting parameters A1, B1, C1 and A2, B2, C2 are processed according to the blood volume information, reflectivity, speed and acceleration of the blood sample, to improve a fitting degree of the cell metabolic signal.

For example, in an embodiment, the above fitting parameters include at least one of:
the blood volume of the blood sample;
a result of calculating the first-order derivatives of the detection curve; or
a result of calculating the second-order derivatives of the detection curve.

In an embodiment, the discrete data is fitted by using multiple different fitting functions respectively, to obtain multiple candidate metabolism curves and fitting degrees thereof, and a candidate metabolism curve with a highest fitting degree is selected from the multiple candidate metabolism curves, as the metabolism curve. In another embodiment, the multiple candidate metabolism curves may also be fused through e.g., averaging, weighted summation, or other methods, and then the metabolism curve may be obtained according to calculated results.

In an embodiment, the controller 140 compares each candidate metabolism curve with the detection curve, calculates a fitting degree of each candidate metabolism curve, and selects a curve of which a fitting degree meets a preset condition from the multiple candidate metabolism curves according to calculated fitting degrees, as a final metabolism curve.

According to the above descriptions, in an embodiment, a fitting function corresponding to an optimal fitting degree may change due to update of the detection signal, for example, the logarithmic fitting function has an optimal fitting degree at a previous time, while the quadratic fitting function has an optimal fitting degree due to update of the detection signal at a next time. Therefore, each time the fitting function is updated, the fitting degree of each fitting function is re-judged and a best fitting function is selected.

The fitting degree of each candidate metabolism curve may be calculated in the following manners. In an embodiment, each candidate metabolism curve is compared with the detection curve based on a Levenberg-Marquardt algorithm, to obtain the fitting degree of each candidate metabolism curve. In another embodiment, the fitting degree may be calculated by using a following fitting degree calculation formula.

$${R}^{2} = 1 - \frac{{Σ ( {Y}_{actual} - {Y}_{predict} )}^{2}}{{Σ ( {Y}_{actual} - {Y}_{m e a n} )}^{2}} ,$$

here R is the fitting degrees, Y_{actual} is a detection signal curve, Y_{predict} is the candidate metabolism curve, and Yₘₑₐₙ is a mean value of the detection signal curve.

In an embodiment, the controller 140 may also perform pre-processing on the detection signal obtained by the detection module 120, to obtain the first information.

In an embodiment, the detection signal may be pre-processed by using median filtering, for example, the detection signal may be processed by using a following algorithm.

y=med{X_{i-L}, X_{i-L+1}, . . . , Xᵢ}, L is a window width, here Xᵢ is a detected numerical value (such as reflectivity) at different culture times, med is taking a median value after sorting L numerical values in an ascending order, if a length of L is an odd number, the median value is directly taken; if the length of L is an even number, a mean value of two numerical values at the middle is taken.

In another embodiment, data of the reflectivity may be processed by using mean filtering, for example, the detection signal may be processed by using a following algorithm.

$$y = \frac{1}{L} {Σ}_{i}^{i - L} {X}_{i} ,$$

here Xᵢ is a detected numerical value (such as reflectivity) at different culture times, and L is a window width.

In another embodiment, it is determined whether a data point in the detection signal exceeds N times of a previous data SD, and when the data point exceeds the N times of the previous data SD, the median filtering is used, and when the data point does not exceed the N times of the previous data SD, the mean filtering is used, to ensure smoothness of the curve while effectively filter out abnormal run-out points.

In another embodiment, the above different pre-processing methods may be combined, the mean filtering is firstly used for the detection signal and then the median filtering is used for the detection signal, or the median filtering is firsty used for the detection signal and then the mean filtering is used for the detection signal.

In an embodiment, an operation of calculating first-order derivatives may also be performed on the filtered detection curve to obtain a speed curve, and an operation of calculating second-order derivatives may be performed on the detection curve to obtain an acceleration curve.

In an embodiment, with reference to FIG. 4, judgment is continuously performed on the first information based on the first preset condition, and judgment is continuously performed on the third information based on the second preset condition. If a culture time reaches an end time of the first preset time period and the detection status of the blood sample has not been labeled as “positive”, a prompt of extending culture is sent to a user or the culture is automatically extended. In this embodiment, when neither the first preset condition nor the second preset condition is met due to slow growth of the micro-organisms within the blood sample, the prompt of extending culture may be sent to the user, or the culture time of the blood sample may be automatically extended, and then detection may be performed, here sending the prompt of extending culture to the user or automatically extending the culture time of the blood sample may be performed automatically, or may be performed in response to a set condition (such as a third preset condition mentioned below).

In another embodiment, during culture, judgment is continuously performed on the first information based on the first preset condition, and judgment is continuously performed on the third information based on the second preset condition. If the first information does not meet the first preset condition and the third information does not meet the second preset condition, the detection status of the blood sample is labeled and outputted as “negative so far”. If a culture time reaches an end time of the first preset time period and the detection status of the blood sample is still labeled as “negative so far”, the prompt of extending culture is sent to the user or the culture is automatically extended. In this embodiment, it is different from the previous embodiment in that when the first preset condition and the second preset condition are not met, the detection status of the blood sample is labeled as “negative so far”, and then detection is continuously performed, until positive reporting is made or the culture time reaches the end time of the first preset time period.

In another embodiment, during culture, judgment is continuously performed on the first information based on the first preset condition, and judgment is continuously performed on the third information based on the second preset condition. If a culture time reaches an end time of the first preset time period and the detection status of the blood sample has not been labeled as “positive”, the detection status of the blood sample is labeled and outputted as “negative”. In this embodiment, when a preset maximum culture time is reached, the detection status of the blood sample is labeled and outputted as “negative”, and a negative report may also be outputted (that is, negative reporting is made).

In another embodiment, during culture, judgment is continuously performed on the first information based on the first preset condition, and judgment is continuously performed on the third information based on the second preset condition. If the first information does not meet the first preset condition and the third information does not meet the second preset condition, the detection status of the blood sample is labeled and outputted as “negative so far”. If a culture time reaches an end time of the first preset time period and the detection status of the blood sample is still labeled as “negative so far”, the detection status of the blood sample is labeled and outputted as “negative”. In this embodiment, it is different from the previous embodiment in that when the first preset condition and the second preset condition are not met, the detection status of the blood sample is labeled as “negative so far”, and then detection is continuously performed, until positive reporting is made or the culture time reaches the end time of the first preset time period.

In an embodiment, the controller 140 also sets a third threshold condition when it processes the detection signal. What is different from the second preset condition for positive reporting is that the third preset condition is used as a preset condition for judging whether it needs to extend culture. That is, for a third signal with a slight increasing trend, a reason why it does not conform to the second preset condition may be an insufficient culture time, such that the third signal may still not reach the second preset condition after reaching the preset maximum culture time. In this case, if negative reporting is made, a false-negative result may occur. In this regard, when it is determined that the third signal conforms to the third preset condition, the prompt of extending culture is sent to the user or the culture time of the blood sample is automatically extended, and the prompt may be sent when the preset maximum culture time is reached. That is, it may be judged whether the third signal conforms to the third preset condition when a culture time reaches the preset maximum culture time, and if the third signal conforms to the third preset condition, the prompt of extending culture is sent to the user or the culture time of the blood sample is automatically extended; if the third signal does not conform to the third preset condition, the detection status of the blood sample is labeled as “negative”.

For example, in an embodiment, if a culture time reaches an end time of the first preset time period, and the first information still does not meet the first preset condition, and the third information still does not meet the second preset condition but meets the third preset condition, the prompt of extending culture is sent to the user or the culture is automatically extended.

For another example, in another embodiment, if a culture time reaches an end time of the first preset time period, and the first information still does not meet the first preset condition, and the third information still does not meet the second preset condition and does not meet the third preset condition, the detection status of the blood sample is labeled and outputted as “negative”.

In an embodiment, the third information is a growth curve related to growth of the micro-organisms within the blood sample in the culture substrate of the sample container at different culture times, and combined judgment of the second preset condition and the third preset condition mentioned in the above embodiments may use at least one of the following judgment conditions (1) to (9).
(1) The second preset condition includes: there continuously occurs a first number of numerical values of the growth curve that are equal to or greater than a first threshold, and the third preset condition includes: the numerical values of the growth curve are equal to or greater than a seventh threshold and less than the first threshold. For example, if the third information does not conform to the second preset condition (the third information is less than the first threshold), it indicates that slow-growing microorganism are not detected within the first preset time period; if the third information conforms to the third preset condition (the third information is greater than the first threshold), it indicates that micro-organisms may still be present in the blood sample, and it needs to extend culture to avoid false-negative reporting; if the third information does not conform to the third preset condition (the third information is less than the seventh threshold), it indicates that there is a low possibility that micro-organisms are present, the detection status of the blood sample is labeled and outputted as “negative”.
(2) The second preset condition includes: there continuously occurs a first number of numerical values of the growth curve that are equal to or greater than a first threshold, and the third preset condition includes: the number of the numerical values of the growth curve which are continuously equal to or greater than the first threshold is less than the first number. For example, if the third information does not conform to the third preset condition (that is, the number of the numerical values which are continuously equal to or greater than the first threshold reaches or exceeds the first number), it indicates that slow-growing micro-organisms may be present within the first preset time period, and it needs to extend culture to avoid false-negative reporting; if the third information conforms to the third preset condition (that is, the number of the numerical values which are continuously equal to or greater than the first threshold is less than the first number), it indicates that there is a low possibility that micro-organisms are present, the detection status of the blood sample is labeled and outputted as “negative”.
(3) The second preset condition includes: a time interval in which numerical values of the growth curve are continuously equal to or greater than a first threshold is equal to or longer than a first time interval threshold, and the third preset condition includes: the time interval in which the numerical values of the growth curve are continuously equal to or greater than the first threshold is shorter than the first time interval threshold. For example, if the third information does not conform to the third preset condition (that is, the time interval in which the numerical values are continuously equal to or greater than the first threshold reaches or exceeds the first time interval threshold), it indicates that slow-growing micro-organisms may be present within the first preset time period, and it needs to extend culture to avoid false-negative reporting; if the third information conforms to the third preset condition (that is, the time interval in which the numerical values are continuously equal to or greater than the first threshold is shorter than the first time interval threshold), it indicates that there is a low possibility that micro-organisms are present, the detection status of the blood sample is labeled and outputted as “negative”.
(4) The second preset condition includes: there continuously occurs a second number of first-order derivatives of the growth curve that are equal to or greater than a second threshold, and the third preset condition includes: the first-order derivatives of the growth curve are equal to or greater than an eighth threshold and less than the second threshold. For example, if the third information does not conform to the third preset condition (that is, a second number of the first-order derivatives which reaches or exceeds the second threshold continuously occur), it indicates that slow-growing micro-organisms may be present within the first preset time period, and it needs to extend culture to avoid false-negative reporting; if the third information conforms to the third preset condition (that is, the first-order derivatives are equal to or greater than the eighth threshold and less than the second threshold), it indicates that there is a low possibility that micro-organisms are present, the detection status of the blood sample is labeled and outputted as “negative”.
(5) The second preset condition includes: there continuously occurs a second number of first-order derivatives of the growth curve that are equal to or greater than a second threshold, and the third preset condition includes: a number of the first-order derivatives of the growth curve which are continuously equal to or greater than the second threshold is less than the second number. If the third information does not conform to the third preset condition (that is, a number of the first-order derivatives occur which are equal to or greater than the second threshold reaches or exceeds the second number), it indicates that slow-growing micro-organisms may be present within the first preset time period, and it needs to extend culture to avoid false-negative reporting; if the third information conforms to the third preset condition (that is, the number of the first-order derivatives occur which are equal to or greater than the second threshold is less than the second number), it indicates that there is a low possibility that micro-organisms are present, the detection status of the blood sample is labeled and outputted as “negative”.
(6) The second preset condition includes: a time interval in which a first-order derivatives of the growth curve are continuously equal to or greater than a second threshold is equal to or longer than a second time interval threshold, and the third preset condition includes: the time interval in which the first-order derivatives of the growth curve are continuously equal to or greater than the second threshold is shorter than the second time interval threshold. For example, if the third information does not conform to the third preset condition (that is, the time interval in which the first-order derivatives are continuously equal to or greater than the second threshold reaches or exceeds the second time interval threshold), it indicates that slow-growing micro-organisms may be present within the first preset time period, and it needs to extend culture to avoid false-negative reporting; if the third information conforms to the third preset condition (that is, the time interval in which the first-order derivatives are continuously equal to or greater than the second threshold is shorter than the second time interval threshold), it indicates that there is a low possibility that micro-organisms are present, the detection status of the blood sample is labeled and outputted as “negative”.
(7) The second preset condition includes: there continuously occurs a third number of second-order derivatives of the growth curve that are equal to or greater than a third threshold, and the third preset condition includes: the second-order derivatives of the growth curve are equal to or greater than a ninth threshold and less than the third threshold. For example, if the third information does not conform to the third preset condition (that is, a third number of the second-order derivatives which reach or exceed the third threshold continuously occur), it indicates that slow-growing micro-organisms may be present within the first preset time period, and it needs to extend culture to avoid false-negative reporting; if the third information conforms to the third preset condition (that is, the second-order derivatives are equal to or greater than the ninth threshold and less than the third threshold), it indicates that there is a low possibility that micro-organisms are present, the detection status of the blood sample is labeled and outputted as “negative”.
(8) The second preset condition includes: there continuously occurs a third number of second-order derivatives of the growth curve that are equal to or greater than a third threshold, and the third preset condition includes: a number of the second-order derivatives of the growth curve which are continuously equal to or greater than the third threshold is less than the third number. For example, if the third information does not conform to the third preset condition (that is, a number of the second-order derivatives which are equal to or greater than the third threshold reaches or exceeds the third number), it indicates that slow-growing micro-organisms may be present within the first preset time period, and it needs to extend culture to avoid false-negative reporting; if the third information conforms to the third preset condition (that is, the number of the second-order derivatives which are equal to or greater than the third threshold is less than the third number), it indicates that there is a low possibility that micro-organisms are present, the detection status of the blood sample is labeled and outputted as “negative”.
(9) The second preset condition includes: a time interval in which second-order derivatives of the growth curve are continuously equal to or greater than a third threshold is equal to or longer than a third time interval threshold, and the third preset condition includes: a time interval in which second-order derivatives of the growth curve are continuously equal to or greater than the third threshold is shorter than the third time interval threshold. For example, if the third information does not conform to the third preset condition (that is, the time interval in which the second-order derivatives are continuously equal to or greater than the third threshold reaches or exceeds the third time interval threshold), it indicates that slow-growing micro-organisms may be present within the first preset time period, and it needs to extend culture to avoid false-negative reporting; if the third information conforms to the third preset condition (that is, the time interval in which the second-order derivatives are continuously equal to or greater than the third threshold is shorter than the third time interval threshold), it indicates that there is a low possibility that micro-organisms are present, the detection status of the blood sample is labeled and outputted as “negative”.

In an embodiment, when triggering a condition that requires extending a culture time of the blood sample, the controller 140 may select a prompt of extension according to user configuration, which prompts the user to determine whether to extend the culture time, or directly and automatically extend the culture time. For example, based on an extension parameter preset or input by the user, the controller 140 automatically extends the culture time of the blood sample to a culture time corresponding to the extension parameter if the culture time reaches the end time of the first preset time period. In an embodiment, it may be determined from the extension parameter that the culture time of the blood sample is to be automatically extended, and then the culture time of the blood sample is automatically extended according to the extension parameter.

In another embodiment, the extension parameter may also be a selection setting of an extension mode. For example, when the extension parameter is a first setting of an automatic extension mode, extension is automatically performed when the condition that requires extending a culture time of the blood sample is triggered; and when the extension parameter is a second setting of a non-automatic extension mode, the prompt of extending culture is sent to the user when the condition that requires extending the culture time of the blood sample is triggered, which prompts the user to input or confirm the culture extension time.

In an embodiment, the operation of sending the prompt of extending culture to the user, includes one of the following contents.

The controller controls the display to display the prompt of extending culture. Preferably, the controller controls the display to display an interface of extending culture, the interface of extending culture is provided with a first control for the user to select whether the culture is extended, the controller is further configured to acquire an interactive operation of the user on the first control to select to perform an extended culture of the blood sample, or to select to abandon the extended culture of the blood sample. Preferably, the interface of extending culture is further provided with a second control for the user to set an extension parameter, the controller is further configured to acquire the extension parameter input by the user, and to perform the extended culture of the blood sample based on the extension parameter.

Or, the blood culture detection system further includes a prompt light, the controller sends the prompt of extending culture to the user through the prompt light.

Or, the blood culture detection system further includes a loudspeaker, the controller sends the prompt of extending culture to the user through the loudspeaker.

Or, the controller is further configured to send the prompt of extending culture to the user through a user terminal or a server.

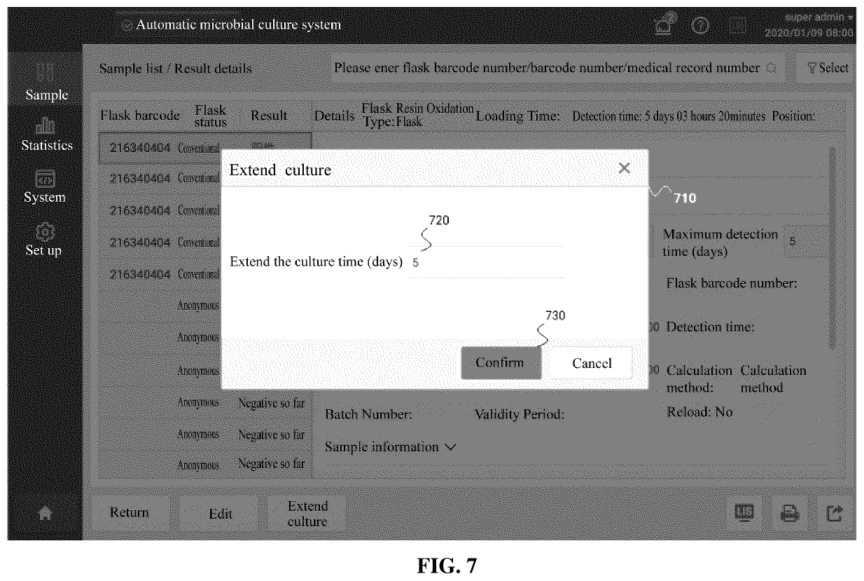

For example, with reference to FIG. 7, an interface of extending culture 710 is displayed on the display, the interface of extending culture 710 includes a second control 720 and a first control 730, and the controller is further configured to acquire the user's operation on the first control 730, to perform the extended culture of the blood sample. For example, the culture time extension (days) is displayed in the interface shown in FIG. 7, and a default extension is 5 days in an embodiment, and the user may click the first control 730 to confirm the extended culture.

The controller is further configured to receive an extended time parameter input by the user, and to perform the extended culture of the blood sample according to the extended time parameter. With reference to FIG. 7, the user may modify the default extended time in an input box of the second control 720, and then click the first control 730 to confirm the extended culture.

In an embodiment, the prompt of extending culture may also be performed in a multi-mode manner by combining manners of performing the prompt of extending culture in any one of the above embodiments.

In an embodiment, within an extended culture time period, the detection signal of the blood sample is continuously acquired and processed to obtain the first information, the second information is continuously acquired, and the third information is obtained based on the first information and the second information. The detection status of the blood sample is labeled and outputted as “positive”, if the first information meets the first preset condition within the extended culture time period. The detection status of the blood sample is labeled and outputted as “positive”, if the third information meets the second preset condition within the extended culture time period. The detection status of the blood sample is labeled and outputted as “negative”, if the first information does not meet the first preset condition and the third information does not meet the second preset condition when the culture time reaches an end time of the extended culture time period. For example, the culture extension is 5 days, if the first preset condition or the second preset condition is met in the period of 5 days, positive reporting is made; and if the first preset condition and the second preset condition are still not met after the fifth day, negative reporting is made.

According to a second aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container; and
obtain third information related to growth of the micro-organisms within the blood sample in the culture substrate of the sample container, based on the first information and the second information.

In some embodiments of the disclosure, the controller acquires and processes the detection signal of the blood sample to obtain the first information, while acquires the second information related to metabolism of the blood cells in the culture substrate, and obtains the third information related to growth of the micro-organisms based on the first information and the second information, such that growth states of the micro-organisms after removing the influence of metabolism of the blood cells can be obtained and observed according to the third information, to provide a more reliable basis for doctors' diagnoses and analysis of the blood culture detection system.

In some embodiments of the disclosure, the first information is a detection curve generated based on detection signals of the blood sample at different culture times, the second information is a metabolism curve related to metabolism of the blood cells within the blood sample in the culture substrate of the sample container at different culture times, and the third information is a growth curve related to growth of the micro-organisms within the blood sample in the culture substrate of the sample container at different culture times. The operation of obtaining the third information related to growth of the micro-organisms within the blood sample in the culture substrate of the sample container, based on the first information and the second information, includes the following contents.

The detection curve is processed according to the metabolism curve, to obtain the growth curve. Preferably, the metabolism curve is subtracted from the detection curve, to obtain the growth curve.

In some embodiments of the disclosure, the second information is a metabolism curve related to metabolism of the blood cells within the blood sample in the culture substrate of the sample container at different culture times. The operation of acquiring the second information related to metabolism of the blood cells within the blood sample in the culture substrate of the sample container, includes the following contents.

The first information is a detection curve generated based on detection signals of the blood sample at different culture times, and the metabolism curve is obtained by performing a matching process based on the detection curve.

Or, the first information is discrete data obtained based on the detection signals of the blood sample at different culture times, and the metabolism curve is obtained by fitting the discrete data.

In some embodiments of the disclosure, the controller is further configured to:
analyze the third information and obtain an analysis result, label and output a detection status of the blood sample as “positive” based on the analysis result, or label and output the detection status of the blood sample as “negative so far” based on the analysis result, or label and output the detection status of the blood sample as “negative” based on the analysis result, or send a prompt of extending culture to a user based on the analysis result, or automatically extend culture of the blood sample based on the analysis result.

And/or, the blood culture detection system further includes a display, and the controller is further configured to:
control the display to display the third information.

In some embodiments of the disclosure, the first information is a detection curve generated based on detection signals of the blood sample at different culture times, the second information is a metabolism curve related to metabolism of the blood cells within the blood sample in the culture substrate of the sample container at different culture times, and the third information is a growth curve related to growth of the micro-organisms within the blood sample in the culture substrate of the sample container at different culture times. The operation of controlling the display to display the third information, includes the following contents.

The display is controlled to display the detection curve, the metabolism curve and the growth curve on a same screen; preferably, the display is controlled to display the detection curve, the metabolism curve and the growth curve in a same coordinate system.

Specific implementations of the blood culture detection system according to the second aspect of the embodiments of the disclosure may refer to the blood culture detection system according to the first aspect, and any implementation of the blood culture detection system according to the first aspect may be directly introduced into the blood culture detection system of this embodiment, which will not be repeatedly elaborated here.

According to a third aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information; and
send a prompt of extending culture to a user or automatically extend culture, according to the third information.

In some embodiments of the disclosure, the controller acquires and processes the detection signal of the blood sample to obtain the first information, while acquires the second information related to metabolism of the blood cells in the culture substrate, obtains the third information related to growth of the micro-organisms based on the first information and the second information, and sends the prompt of extending culture to the user or automatically extends culture according to the third information. Whether it needs to extend culture time is intelligently judged through analyzing growth states of the micro-organisms after removing the influence of metabolism of the blood cells, which can effectively solve the technical false-negative problem in some implementations caused by the slow-growing micro-organisms cannot being detected as positive within the maximum culture duration due to their slow growth, avoid a false-negative detection result of the slow-growing micro-organisms due to early termination of culture that may be caused by a traditional fixed culture time, significantly improve a detection rate of the slow-growing micro-organisms by the blood culture detection system, and provide a more accurate and reliable detection result for clinical diagnosis.

In some embodiments of the disclosure, the operation of sending the prompt of extending culture to the user or automatically extending the culture, according to the third information, includes the following contents.

If a culture time reaches an end time of a first preset time period, and the third information does not meet a second preset condition but meets a third preset condition, the prompt of extending culture is sent to the user or the culture is automatically extended.

And/or, if the culture time reaches the end time of the first preset time period, and the third information does not meet the second preset condition and does not meet the third preset condition, a detection status of the blood sample is labeled and outputted as “negative”.

In some embodiments of the disclosure, the third information is a growth curve related to growth of the micro-organisms within the blood sample in the culture substrate of the sample container at different culture times.

The second preset condition includes: there continuously occurs a first number of numerical values of the growth curve that are equal to or greater than a first threshold, and the third preset condition includes: the numerical value of the growth curve is equal to or greater than a seventh threshold and less than the first threshold.

Or, the second preset condition includes: there continuously occurs a first number of numerical values of the growth curve that are equal to or greater than a first threshold, and the third preset condition includes: a number of the numerical values of the growth curve which are continuously equal to or greater than the first threshold is less than the first number.

Or, the second preset condition includes: a time interval in which numerical values of the growth curve are continuously equal to or greater than a first threshold is equal to or longer than a first time interval threshold, and the third preset condition includes: the time interval in which the numerical values of the growth curve are continuously equal to or greater than the first threshold is shorter than the first time interval threshold.

Or, the second preset condition includes: there continuously occurs a second number of first-order derivatives of the growth curve that are equal to or greater than a second threshold, and the third preset condition includes: the first-order derivatives of the growth curve are equal to or greater than an eighth threshold and less than the second threshold.

Or, the second preset condition includes: there continuously occurs a second number of first-order derivatives of the growth curve that are equal to or greater than a second threshold, and the third preset condition includes: a number of the first-order derivatives of the growth curve which are equal to or greater than the second threshold is less than the second number.

Or, the second preset condition includes: a time interval in which a first-order derivatives of the growth curve are continuously equal to or greater than a second threshold is equal to or longer than a second time interval threshold, and the third preset condition includes: the time interval in which the first-order derivatives of the growth curve are continuously equal to or greater than the second threshold is shorter than the second time interval threshold.

Or, the second preset condition includes: there continuously occurs a third number of second-order derivatives of the growth curve that are equal to or greater than a third threshold, and the third preset condition includes: the second-order derivatives of the growth curve are equal to or greater than a ninth threshold and less than the third threshold.

Or, the second preset condition includes: there continuously occurs a third number of second-order derivatives of the growth curve that are equal to or greater than a third threshold, and the third preset condition includes: a number of the second-order derivatives of the growth curve which are continuously equal to or greater than the third threshold is less than the third number.

Or, the second preset condition includes: a time interval in which second-order derivatives of the growth curve are continuously equal to or greater than a third threshold is equal to or longer than a third time interval threshold, and the third preset condition includes: a time interval in which the second-order derivative of the growth curve are continuously equal to or greater than the third threshold is shorter than the third time interval threshold.

Specific implementations of the blood culture detection system according to the third aspect of the embodiments of the disclosure may refer to the blood culture detection system according to the first aspect, and any implementation of the blood culture detection system according to the first aspect may be directly introduced into the blood culture detection system of this embodiment, which will not be repeatedly elaborated here.

According to a fourth aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and generate a detection signal of the blood sample.

The controller is configured to:
acquire and process the detection signal of the blood sample, so as to obtain first information of the blood sample;
if the first information meets a first preset condition within a first preset time period, label and output a detection status of the blood sample as “positive”, here the first preset condition is set based on an influence of metabolism of micro-organisms within the blood sample in the culture substrate on the detection signal of the blood sample; if the first information meets a fourth preset condition within the first preset time period, label and output the detection status of the blood sample as “positive”, here the fourth preset condition is set based on an influence of metabolism of blood cells within the blood sample in the culture substrate on the detection signal of the blood sample; or
if a culture time reaches an end time of the first preset time period, and the first information still does not meet the first preset condition but meets the fourth preset condition, send a prompt of extending culture to a user or automatically extend culture; and/or
if a culture time reaches an end time of the first preset time period, and the first information still does not meet the first preset condition and does not meet the fourth preset condition, label and output the detection status of the blood sample as “negative”, here the fourth preset condition is set based on an influence of metabolism of blood cells within the blood sample in the culture substrate on the detection signal of the blood sample.

In some embodiments of the disclosure, the controller acquires and processes the detection signal of the blood sample to obtain the first information, and judges the first information according to the first preset condition to detect normal-growing micro-organisms, and further judges the first information according to the fourth preset condition which is set based on the influence of metabolism of blood cells within the blood sample in the culture substrate on the detection signal of the blood sample, to detect slow-growing micro-organisms, which can effectively solve the false-negative problem in some implementations caused by the slow-growing micro-organisms cannot being detected as positive within the maximum culture duration due to their slow growth, significantly improve accuracy of the blood culture detection system, and reduce false-negative results.

In some embodiments of the disclosure, the first information is judged according to the first preset condition, to detect the normal-growing micro-organisms and label them as “positive” in time. In another embodiment, when a culture time reaches an end time of the first preset time period, the first information is judged according to the fourth preset condition; if the fourth preset condition is met, the prompt of extending culture is sent to the user or the culture is automatically extended; and if the fourth preset condition is not met, the micro-organisms are labeled as “negative”. Early termination of culture that may be caused by a traditional fixed culture time is avoided by an intelligent extension culture mechanism, which can effectively solve the technical false-negative problem in some implementations caused by the slow-growing micro-organisms cannot being detected as positive within the maximum culture duration due to their slow growth, significantly improve a detection rate of the slow-growing micro-organisms by the blood culture detection system, reduce false-negative results, and provide a more accurate and reliable detection result for clinical diagnosis.

In some embodiments of the disclosure, if the first information meets the fourth preset condition within the first preset time period, the operation of labeling and outputting the detection status of the blood sample as “positive”, includes the following contents.

The first information within the first preset time period is input to a machine learning model trained in advance, to perform judgment, and if the judgment confirms that the first information meets the fourth preset condition, the detection status of the blood sample is labeled and outputted as “positive”. The machine learning model is obtained by training with a training set containing at least information related to metabolism of the blood cells within the blood sample in the culture substrate.

Specific implementations of the blood culture detection system according to the fourth aspect of the embodiments of the disclosure may refer to the blood culture detection system according to the first aspect, and any implementation of the blood culture detection system according to the first aspect may be directly introduced into the blood culture detection system of this embodiment, which will not be repeatedly elaborated here. The fourth preset condition may refer to descriptions of the second preset condition in any one of the foregoing embodiments, and the descriptions of the second preset condition may be applied to the fourth preset condition of this embodiment.

According to a fifth aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
if the first information meets a preset model within a first preset time period, label and output a detection status of the blood sample as “positive”; or
if a culture time reaches an end time of the first preset time period, and the first information does not meet the preset model, label the detection status of the blood sample as “negative” or “negative so far”; or
if the first information within the first preset time period conforms to a preset model, send a prompt of extending culture to a user or automatically extend culture, here the preset model is a model established based on an influence of metabolism of blood cells within the blood sample in the culture substrate on the detection signal of the blood sample.

In some embodiments of the disclosure, the controller acquires and processes the detection signal of the blood sample to obtain the first information, and judges the first information by using the preset model which is established based on the influence of metabolism of the blood cells within the blood sample in the culture substrate on the detection signal of the blood sample. When the first information meets the preset model, the detection status is labeled and outputted as “positive”. When the culture time reaches the end time of the first preset time period, and the first information does not meet the preset model, the detection status is labeled as “negative” or “negative so far”. Therefore, it can effectively solve the false-negative problem in some implementations caused by the slow-growing micro-organisms cannot being detected as positive within the maximum culture duration due to their slow growth, significantly improve accuracy of identifying different types of micro-organisms by the blood culture detection system through the preset model where the influence of metabolism of the blood cells on the detection signal is considered, reduce false-negative results, and provide a more accurate and reliable detection result for clinical diagnosis.

In some embodiments of the disclosure, the first information is judged by using the preset model which is established based on the influence of metabolism of the blood cells within the blood sample in the culture substrate on the detection signal of the blood sample, and when the first information within the first preset time period conforms to the preset model, the prompt of extending culture is sent to the user or the culture is automatically extended. Requirements of extending the culture are intelligently judged to avoid the problem that the slow-growing micro-organisms cannot be identified due to early termination of culture that may be caused by a traditional fixed culture time, can may significantly improve a detection rate of the slow-growing micro-organisms by the blood culture detection system, reduce false-negative results, and provide a more accurate and reliable detection result for clinical diagnosis.

Specific implementations of the blood culture detection system according to the fifth aspect of the embodiments of the disclosure may refer to the blood culture detection system according to the first aspect, and any implementation of the blood culture detection system according to the first aspect may be directly introduced into the blood culture detection system of this embodiment, which will not be repeatedly elaborated here.

According to a sixth aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information; and
if the first information does not meet a first preset condition within a first preset time period, and the third information does not meet a second preset condition within the first preset time period, label and output the detection status of the blood sample as “negative” or “negative so far”.

In some embodiments of the disclosure, the controller acquires and processes the detection signal of the blood sample to obtain the first information, acquires the second information related to metabolism of the blood cells in the culture substrate, and obtains the third information related to growth of the micro-organisms based on the first information and the second information. On one hand, the first information is judged according to the first preset condition, to detect normal-growing micro-organisms. On the other hand, the third information obtained based on the first information and the second information is judged according to the second preset condition. When the first information does not meet the first preset condition and the third information does not meet the second preset condition, the detection status is labeled and outputted as “negative” or “negative so far”. Therefore, it can improve accuracy of the negative status by removing the influence of metabolism of the blood cells, and provide a more accurate and credible detection result for clinical diagnosis.

In some embodiments of the disclosure, if the first information does not meet the first preset condition within the first preset time period, and the third information does not meet the second preset condition within the first preset time period, the operation of labeling and outputting the detection status of the blood sample as “negative so far”, includes one of the following contents.

During culture within the first preset time period, judgment is continuously performed on the first information based on the first preset condition, and judgment is continuously performed on the third information based on the second preset condition. If the first information does not meet the first preset condition and the third information does not meet the second preset condition, the detection status of the blood sample is labeled and outputted as “negative so far”. If a culture time reaches an end time of the first preset time period and the detection status of the blood sample is still labeled as “negative so far”, a prompt of extending culture is sent to a user or the culture is automatically extended.

Or, during culture within the first preset time period, judgment is continuously performed on the first information based on the first preset condition, and judgment is continuously performed on the third information based on the second preset condition. If the first information does not meet the first preset condition and the third information does not meet the second preset condition, the detection status of the blood sample is labeled and outputted as “negative so far”. If a culture time reaches an end time of the first preset time period and the detection status of the blood sample is still labeled as “negative so far”, the detection status of the blood sample is labeled and outputted as “negative”.

In some embodiments of the disclosure, the controller is further configured to:
if a culture time reaches an end time of the first preset time period, the first information still does not meet the first preset condition, and the third information still does not meet the second preset condition but meets a third preset condition, send a prompt of extending culture to a user or automatically extend culture; and/or
if an culture time reaches an end time of the first preset time period, the first information still does not meet the first preset condition, and the third information still does not meet the second preset condition and does not meet the third preset condition, label and output the detection status of the blood sample as “negative”.

In some embodiments of the disclosure, the third information is a growth curve related to growth of the micro-organisms within the blood sample in the culture substrate of the sample container at different culture times.

The second preset condition includes: there continuously occurs a first number of numerical values of the growth curve that are equal to or greater than a first threshold, and the third preset condition includes: the numerical values of the growth curve are equal to or greater than a seventh threshold and less than the first threshold.

Or, the second preset condition includes: there continuously occurs a first number of numerical values of the growth curve that are equal to or greater than a first threshold, and the third preset condition includes: a number of the numerical values of the growth curve which are continuously equal to or greater than the first threshold is less than the first number.

Or, the second preset condition includes: a time interval in which a numerical value of the growth curve is continuously equal to or greater than a first threshold is equal to or longer than a first time interval threshold, and the third preset condition includes: the time interval in which the numerical values of the growth curve are continuously equal to or greater than the first threshold is shorter than the first time interval threshold.

Or, the second preset condition includes: there continuously occurs a second number of first-order derivatives of the growth curve that are equal to or greater than a second threshold, and the third preset condition includes: the first-order derivatives of the growth curve are equal to or greater than an eighth threshold and less than the second threshold.

Or, the second preset condition includes: there continuously occurs a second number of first-order derivatives of the growth curve that are equal to or greater than a second threshold, and the third preset condition includes: a number of the first-order derivatives of the growth curve which are equal to or greater than the second threshold is less than the second number.

Or, the second preset condition includes: a time interval in which first-order derivatives of the growth curve are continuously equal to or greater than a second threshold is equal to or longer than a second time interval threshold, and the third preset condition includes: the time interval in which the first-order derivatives of the growth curve are continuously equal to or greater than the second threshold is shorter than the second time interval threshold.

Or, the second preset condition includes: there continuously occurs a third number of second-order derivatives of the growth curve that are equal to or greater than a third threshold, and the third preset condition includes: the second-order derivatives of the growth curve are equal to or greater than a ninth threshold and less than the third threshold.

Or, the second preset condition includes: there continuously occurs a third number of second-order derivatives of the growth curve that are equal to or greater than a third threshold, and the third preset condition includes: a number of the second-order derivatives of the growth curve which are continuously equal to or greater than the third threshold is less than the third number.

Or, the second preset condition includes: a time interval in which a second-order derivatives of the growth curve are continuously equal to or greater than a third threshold is equal to or longer than a third time interval threshold, and the third preset condition includes: a time interval in which the second-order derivatives of the growth curve which are continuously equal to or greater than the third threshold is shorter than the third time interval threshold.

In some embodiments of the disclosure, the controller is further configured to:
within an extended culture time period, continuously acquire and process the detection signal of the blood sample, so as to obtain the first information, continuously acquire the second information, and obtain the third information based on the first information and the second information;
label and output the detection status of the blood sample as “positive”, if the first information meets the first preset condition within the extended culture time period;
label and output the detection status of the blood sample as “positive”, if the third information meets the second preset condition within the extended culture time period;
label and output the detection status of the blood sample as “negative”, if the first information does not meet the first preset condition and the third information does not meet the second preset condition when the culture time reaches an end time of the extended culture time period.

Specific implementations of the blood culture detection system according to the sixth aspect of the embodiments of the disclosure may refer to the blood culture detection system according to the first aspect, and any implementation of the blood culture detection system according to the first aspect may be directly introduced into the blood culture detection system of this embodiment, which will not be repeatedly elaborated here.

According to a seventh aspect, an embodiment of the disclosure provides a blood culture detection system, the blood culture detection system includes an incubation module, a detection module and a controller.

The incubation module includes an incubation component and a temperature control component, the incubation component is configured to hold at least one sample container carrying a blood sample and a culture substrate and to culture micro-organisms within the blood sample at a preset temperature, and the temperature control component is configured to regulate the preset temperature.

The detection module includes at least one detection sensor configured to detect the blood sample and to generate a detection signal of the blood sample.

The controller is configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information; and
if the third information conforms to a second preset condition, label and output a detection status of the blood sample as “positive”; or, if the third information does not conform to the second preset condition, label the detection status of the blood sample as “negative so far” or “negative”.

In some embodiments of the disclosure, the controller acquires and processes the detection signal of the blood sample to obtain the first information, acquires the second information related to metabolism of the blood cells in the culture substrate, obtains the third information related to growth of the micro-organisms based on the first information and the second information, and judges the third information obtained based on the first information and the second information according to the second preset condition. In an embodiment, if the third information conforms to the second preset condition, the detection status is labeled and outputted as “positive”; in another embodiment, if the third information does not conform to the second preset condition, the detection status is labeled as “negative so far” or “negative”. The micro-organisms are detected and judged based on the third information after removing the influence of metabolism of the blood cells, which can effectively solve the false-negative problem in some implementations caused by the slow-growing micro-organisms cannot being detected as positive within the maximum culture duration due to their slow growth, significantly improve accuracy of detecting the micro-organisms by the blood culture detection system, reduce false-negative results, and provide a more accurate and credible detection result for clinical diagnosis.

In some embodiments of the disclosure, the first information is a detection curve generated based on detection signals of the blood sample at different culture times, the second information is a metabolism curve related to metabolism of the blood cells within the blood sample in the culture substrate of the sample container at different culture times, and the third information is a growth curve related to growth of the micro-organisms within the blood sample in the culture substrate of the sample container at different culture times. The operation of obtaining the third information related to growth of the micro-organisms within the blood sample in the culture substrate of the sample container, based on the first information and the second information, includes the following contents.

The detection curve is processed according to the metabolism curve, to obtain the growth curve. Preferably, the metabolism curve is subtracted from the detection curve, to obtain the growth curve.

In some embodiments of the disclosure, the second preset condition includes at least one of the following conditions.

There continuously occurs a first number of numerical values of the growth curve that are equal to or greater than a first threshold, or a time interval in which numerical values of the growth curve are continuously equal to or greater than a first threshold is equal to or longer than a first time interval threshold.

Or, there continuously occurs a second number of first-order derivatives of the growth curve that are equal to or greater than a second threshold, or a time interval in which first-order derivatives of the growth curve are continuously equal to or greater than a second threshold is equal to or longer than a second time interval threshold.

Or, there continuously occurs a third number of second-order derivatives of the growth curve that are equal to or greater than a third threshold, or a time interval in which second-order derivatives of the growth curve are continuously equal to or greater than a third threshold is equal to or longer than a third time interval threshold.

In some embodiments of the disclosure, the controller is further configured to:
if a culture time reaches an end time of a first preset time period, and the third information does not meet the second preset condition but meets a third preset condition, send a prompt of extending culture to a user or automatically extend culture; and/or
if a culture time reaches an end time of the first preset time period, and the third information does not meet the second preset condition and does not meet the third preset condition, label and output the detection status of the blood sample as “negative”.

In some embodiments of the disclosure, if the third information does not conform to the second preset condition, the operation of labeling the detection status of the blood sample as “negative so far”, includes one of the following contents.

During culture within a first preset time period, judgment is continuously performed on the third information based on the second preset condition. If the third information does not meet the second preset condition, the detection status of the blood sample is labeled and outputted as “negative so far”. If a culture time reaches an end time of the first preset time period and the detection status of the blood sample is still labeled as “negative so far”, a prompt of extending culture is sent to a user or the culture is automatically extended.

Or, during culture within the first preset time period, judgment is continuously performed on the third information based on the second preset condition. If the third information does not meet the second preset condition, the detection status of the blood sample is labeled and outputted as “negative so far”. If a culture time reaches an end time of the first preset time period and the detection status of the blood sample is still labeled as “negative so far”, the detection status of the blood sample is labeled and outputted as “negative”.

In some embodiments of the disclosure, the controller is further configured to:
within an extended culture time period, continuously acquire and process the detection signal of the blood sample, so as to obtain the first information, continuously acquire the second information, and obtain the third information based on the first information and the second information;
label and output the detection status of the blood sample as “positive”, if the third information meets the second preset condition within the extended culture time period;
label and output the detection status of the blood sample as “negative”, if the third information does not meet the second preset condition when the culture time reaches an end time of the extended culture time period.

Specific implementations of the blood culture detection system according to the seventh aspect of the embodiments of the disclosure may refer to the blood culture detection system according to the first aspect, and any implementation of the blood culture detection system according to the first aspect may be directly introduced into the blood culture detection system of this embodiment, which will not be repeatedly elaborated here.

According to an eighth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

If the first information meets a first preset condition within a first preset time period, a detection status of the blood sample is labeled and outputted as “positive”.

If the third information meets a second preset condition within the first preset time period, the detection status of the blood sample is labeled and outputted as “positive”.

Further specific implementations of the blood sample detection method according to the eighth aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here.

According to a ninth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

The display is controlled to display the third information.

Further specific implementations of the sample detection method according to the ninth aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here.

According to a tenth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

A prompt of extending culture is sent to a user or the culture is automatically extended, according to the third information.

Further specific implementations of the blood sample detection method according to the tenth aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here.

According to an eleventh aspect, an embodiment of the disclosure provides a sample detection method, the sample detection method includes the following operations.

A detection module acquires a detection signal of a blood sample, and processes the detection signal to obtain first information of the blood sample.

If the first information meets a first preset condition within a first preset time period, a detection status of the blood sample is labeled and outputted as “positive”, here the first preset condition is set based on an influence of metabolism of micro-organisms within the blood sample in a culture substrate on the detection signal of the blood sample; and if the first information meets a fourth preset condition within the first preset time period, the detection status of the blood sample is labeled and outputted as “positive”, here the fourth preset condition is set based on an influence of metabolism of blood cells within the blood sample in the culture substrate on the detection signal of the blood sample.

Or, if a culture time reaches an end time of the first preset time period, and the first information still does not meet the first preset condition but meets the fourth preset condition, a prompt of extending culture is sent to a user or the culture is automatically extended.

And/or, if a culture time reaches an end time of the first preset time period, and the first information still does not meet the first preset condition and does not meet the fourth preset condition, the detection status of the blood sample is labeled and outputted as “negative”, here the fourth preset condition is set based on an influence of metabolism of blood cells within the blood sample in the culture substrate on the detection signal of the blood sample.

Further specific implementations of the blood sample detection method according to the eleventh aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here. The fourth preset condition may refer to descriptions of the second preset condition in any one of the foregoing embodiments, and the descriptions of the second preset condition may be applied to the fourth preset condition of this embodiment.

According to a twelfth aspect, an embodiment of the disclosure provides a sample detection method, the sample detection method includes the following operations.

A detection module acquires a detection signal of a blood sample, and processes the detection signal to obtain first information of the blood sample.

If the first information meets a preset model within a first preset time period, a detection status of the blood sample is labeled and outputted as “positive”.

Or, if a culture time reaches an end time of the first preset time period, and the first information does not meet the preset model, the detection status of the blood sample is labeled as “negative” or “negative so far”.

Or, if the first information within the first preset time period conforms to a preset model, a prompt of extending culture is sent to a user or the culture is automatically extended, here the preset model is a model established based on an influence of metabolism of blood cells within the blood sample in a culture substrate on the detection signal of the blood sample.

Further specific implementations of the blood sample detection method according to the twelfth aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here.

According to a thirteenth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

If the first information does not meet a first preset condition within a first preset time period, and the third information does not meet a second preset condition within the first preset time period, the detection status of the blood sample is labeled and outputted as “negative” or “negative so far”.

Further specific implementations of the blood sample detection method according to the thirteenth aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here.

According to a fourteenth aspect, an embodiment of the disclosure provides a sample detection method, the sample detection method includes the following operations.

A detection module acquires a detection signal of a blood sample, and processes the detection signal to obtain first information of the blood sample.

The detection module acquires second information related to metabolism of blood cells within the blood sample in a culture substrate of the sample container.

The detection module obtains third information related to growth of micro-organisms within the blood sample in the culture substrate of the sample container, based on the first information and the second information.

If the third information conforms to a second preset condition, a detection status of the blood sample is labeled and outputted as “positive”; or, if the third information does not conform to the second preset condition, the detection status of the blood sample is labeled as “negative so far” or “negative”.

Further specific implementations of the blood sample detection method according to the fourteenth aspect of the embodiments of the disclosure may refer to relevant operations of the controller 140 in the blood culture detection system according to the first aspect, and any implementation of the blood culture detection system according to the first aspect may be directly introduced into the blood sample detection method of this embodiment, which will not be repeatedly elaborated here.

According to a fifteenth aspect, an embodiment of the disclosure provides an electronic device, the electronic device includes a memory and a control component, the memory stores a computer program, and the sample detection method according to any one of the above embodiments is implemented when the control component executes the computer program.

According to a sixteenth aspect, an embodiment of the disclosure provides a computer storage medium, the computer storage medium has stored thereon a computer program. The computer storage medium is applied to a pipeline system, and the sample detection method according to any one of the above embodiments is implemented when the computer program is executed by a control component.

According to a seventeenth aspect, an embodiment of the disclosure provides a computer program product or a computer program, the computer program product or the computer program includes computer instructions stored in a computer-readable storage medium. A control component of a computer device reads the computer instructions from the computer-readable storage medium, and the control component executes the computer instructions, such that the computer device executes the sample detection method according to any one of the above embodiments.

Terms “first”, “second”, “third”, “fourth” or the like (if any) in the description of the disclosure and the above drawings are intended to distinguish similar objects, and are not necessarily intended to describe a specific order or sequence. It should be understood that data used as such may be interchangeable if appropriate, to describe the embodiments of the disclosure, for example, they may be implemented in an order other than those shown or described here. Furthermore, terms “include” and “have” and any variation thereof are intended to cover a non-exclusive inclusion, for example, processes, methods, systems, products or devices including a series of operations or units are not necessarily limited to those operations or units that are clearly listed, instead, may include other operations or units that are not clearly listed or inherent to these processes, methods, products or devices.

It should be understood that in the disclosure, “at least one” refers to “one or more”, and “multiple” refers to “two or more”. “and/or” is intended to describe an association relationship of associated object, and indicates that there may be three relationships. For example, “A and/or B” may mean three cases: only A exists, only B exists, and both A and B exist, here A and B may be in a singular or plural form. A character “/” usually indicates that anterior and posterior associated objects are in an “or” relationship. “at least one of the following items” or a similar expression thereof refers to any combination of these items, including any combination of single or plural items. For example, at least one of a, b or c may indicate: a, b, c, “a and b”, “a and c”, “b and c”, or “a and b and c”, here a, b, c may be in a singular or plural form.

It should be understood that in the descriptions of the embodiments of the disclosure, the meaning of “multiple” (or multiple items) is “two or more”; “greater than”, “less than”, “exceed” or the like is understood as excluding the number itself, and “above”, “below”, “within” or the like is understood as including the number itself.

In several embodiments according to the disclosure, it should be understood that the disclosed system, device and method may be implemented in other manners. For example, the above device embodiments are schematic only. For example, division of the units is only a logic function division, and other division manners may be adopted in an actual implementation. For example, multiple units or components may be combined or integrated into another system, or some features may be ignored or may not be performed. In addition, coupling or direct coupling or communication connection between displayed or discussed components may be indirect coupling or communication connection of the device or the units implemented through some interfaces, and may be in electrical, mechanical or other forms.

The units described as separate components may be or may not be physically separated, and components displayed as units may be or may not be physical units, that is, they may be located at the same place, or may be distributed to multiple network units. Part or all of the units may be selected to achieve the purpose of the solutions in the embodiments according to actual requirements.

Furthermore, each functional unit in each embodiment of the disclosure may be integrated into a processing unit, or each unit may physically exist separately, or two or more than two units may be integrated into a unit. The above integrated unit may be implemented in a form of hardware or in a form of software functional unit.

When the integrated unit is implemented in a form of software functional unit and is sold or used as an independent product, the integrated unit may be stored in a computer-readable storage medium. Based on such understanding, the technical solutions of the disclosure substantially or parts making contributions to some implementations or all or part of the technical solutions may be embodied in a form of software product, and the computer software product is stored in a storage medium, and includes several instructions configured to enable a computer device (which may be a personal computer, a server, or a network device, etc.) to execute all or part of operations of the method described in each embodiment of the disclosure. The foregoing storage medium includes various media capable of storing program codes, such as a U disk, a mobile hard disk, a Read-Only Memory (ROM), a Random Access Memory (RAM), a magnetic disk, or an optical disk, etc.

It should also be understood that various implementations according to the embodiments of the disclosure may be arbitrarily combined, to achieve different technical effects.

The above descriptions are specific descriptions of preferred implementations of the disclosure, however, the disclosure is not limited the above implementations. Technicians familiar with this field may also make various equivalent modifications or replacements without violating the spirit of the disclosure, and these equivalent modifications or replacements should be included within the scope defined by the claims of the disclosure.

1. A blood culture detection system, comprising:
an incubation module, comprising an incubation component and a temperature control component, wherein the incubation component is configured to hold at least one sample container carrying a blood sample and a culture substrate and to culture micro-organisms within the blood sample at a preset temperature, and the temperature control component is configured to regulate the preset temperature;
a detection module, comprising at least one detection sensor configured to detect the blood sample and to generate a detection signal of the blood sample; and
a controller, configured to:
acquire and process the detection signal of the blood sample, so as to obtain first information of the blood sample;
acquire second information related to metabolism of blood cells within the blood sample in the culture substrate of the sample container;
obtain third information related to growth of the micro-organisms within the blood sample in the culture substrate of the sample container, based on the first information and the second information;
if the first information meets a first preset condition within a first preset time period, label and output a detection status of the blood sample as “positive”;
if the third information meets a second preset condition within the first preset time period, label and output a detection status of the blood sample as “positive”.

2. The blood culture detection system of claim 1, wherein the first preset time period is a minimum culture time required for clinically labeling and outputting a detection status of the blood sample as “negative”.

3. The blood culture detection system of claim 1, wherein the controller configured to acquire second information related to metabolism of blood cells within the blood sample in the culture substrate of the sample container, comprises: the controller configured to:
process a detection signal of the blood sample within a second preset time period, so as to obtain the second information, wherein the first preset time period comprises the second preset time period and is longer than the second preset time period; and
wherein the detection signal of the blood sample within the second preset time period is substantially originated from metabolism of the blood cells within the blood sample in the culture substrate of the sample container.

4. The blood culture detection system of claim 3, wherein the controller configured to process a detection signal of the blood sample within a second preset time period, so as to obtain the second information, comprises: the controller configured to:
process the detection signal of the blood sample within the second preset time period, so as to obtain a processing result, and generate the second information based on the processing result; or
process the detection signal of the blood sample within the second preset time period, so as to obtain a processing result, and obtain the second information by matching from a plurality of pieces of preset information based on the processing result.

5. The blood culture detection system of claim 3, wherein the controller configured to: if the third information meets a second preset condition within the first preset time period, label and output a detection status of the blood sample as “positive”, comprises: the controller configured to:
if the third information meets the second preset condition within a third preset time period, label and output the detection status of the blood sample as “positive”, wherein the first preset time period comprises the third preset time period, the second preset time period does not comprise the third preset time period while may partially coincide with the third preset time period, and a starting point of the second preset time period is earlier than a starting point of the third preset time period.

6. The blood culture detection system of claim 1, wherein the controller configured to acquire second information related to metabolism of blood cells within the blood sample in the culture substrate of the sample container, comprises: the controller configured to:
select the second information from a plurality of pieces of preset information based on a blood volume of the blood sample, wherein the plurality of pieces of preset information are configured to respectively characterize metabolism of blood cells within a plurality of blood samples with different blood volumes in the culture substrate.

7. The blood culture detection system of claim 1, wherein the controller configured to obtain third information related to growth of the micro-organisms within the blood sample in the culture substrate of the sample container, based on the first information and the second information, comprises: the controller configured to:
obtain the third information by removing the second information from the first information.

8. The blood culture detection system of claim 1, wherein the second information comprises one of:
information generated based on the first information;
a piece of preset information;
information determined based on a blood volume of the blood sample;
information selected from a plurality of pieces of preset information; or
information obtained by matching from a plurality of pieces of preset information based on the first information;
and/or,
wherein the third information is a growth curve related to growth of the micro-organisms within the blood sample in the culture substrate of the sample container at different culture times; the controller configured to: if the third information meets a second preset condition within the first preset time period, label and output a detection status of the blood sample as “positive”, comprises: the controller configured to:
if the growth curve meets the second preset condition within the first preset time period, label and output the detection status of the blood sample as “positive”.

9. The blood culture detection system of claim 8, wherein the second preset condition comprises at least one of:
there continuously occurs a first number of numerical values of the growth curve that are equal to or greater than a first threshold, or a time interval in which numerical values of the growth curve are continuously equal to or greater than a first threshold is equal to or longer than a first time interval threshold;
there continuously occurs a second number of first-order derivatives of the growth curve that are equal to or greater than a second threshold, or a time interval in which first-order derivatives of the growth curve are continuously equal to or greater than a second threshold is equal to or longer than a second time interval threshold; and
there continuously occurs a third number of second-order derivatives of the growth curve that are equal to or greater than a third threshold, or a time interval in which second-order derivatives of the growth curve are continuously equal to or greater than a third threshold is equal to or longer than a third time interval threshold.

10. The blood culture detection system of claim 1, wherein the first information is a detection curve generated based on detection signals of the blood sample at different culture times, the second information is a metabolism curve related to metabolism of the blood cells within the blood sample in the culture substrate of the sample container at different culture times, and the third information is a growth curve related to growth of the micro-organisms within the blood sample in the culture substrate of the sample container at different culture times; the controller configured to obtain third information related to growth of the micro-organisms within the blood sample in the culture substrate of the sample container, based on the first information and the second information, comprise: the controller configured to:
process the detection curve according to the metabolism curve, so as to obtain the growth curve, and/or subtracting the metabolism curve from the detection curve, so as to obtain the growth curve;
and/or,
wherein the second information is a metabolism curve related to metabolism of the blood cells within the blood sample in the culture substrate of the sample container at different culture times; the controller configured to acquire second information related to metabolism of the blood cells within the blood sample in the culture substrate of the sample container, comprises: the controller configured to:
when the first information is a detection curve generated based on detection signals of the blood sample at different culture times, obtain the metabolism curve by performing a matching process based on the detection curve; or
when the first information is discrete data obtained based on detection signals of the blood sample at different culture times, obtain the metabolism curve by fitting the discrete data.

11. The blood culture detection system of claim 10, wherein the controller configured to obtain the metabolism curve by performing a matching process based on the detection curve, comprise: the controller configured to:
obtain the metabolism curve by performing the matching process based on the detection curve within a second preset time period, wherein a detection signal of the blood sample within the second preset time period is substantially originated from the metabolism of the blood cells within the blood sample in the culture substrate of the sample container; or
within the second preset time period, obtain the metabolism curve by periodically performing the matching process based on the detection curve; and outside the second preset time period, take at least one metabolism curve obtained within the second preset time period as a metabolism curve to be used currently, wherein the detection signal of the blood sample within the second preset time period is substantially originated from the metabolism of the blood cells within the blood sample in the culture substrate of the sample container; or
obtain the metabolism curve by performing the matching process based on a blood volume of the blood sample and the detection curve; or
obtain the metabolism curve by performing a first-order derivation and/or a second-order derivation on the detection curve and performing the matching process based on a result of the first-order derivation and/or a result of the second-order derivation;
or,
wherein the controller configured to obtain the metabolism curve by fitting the discrete data, comprises: the controller configured to:
obtain the metabolism curve by fitting the discrete data with a fitting function, wherein the fitting function comprises at least one of a logarithmic fitting function and a quadratic fitting function; or
fit the discrete data with a plurality of different fitting functions respectively, so as to obtain a plurality of candidate metabolism curves and their respective fitting degrees; and select, from the plurality of candidate metabolism curves, a candidate metabolism curve with a highest fitting degree as the metabolism curve; or
periodically fit the discrete data during culture, so as to update the metabolism curve.

12. The blood culture detection system of claim 1, wherein the detection sensor is an optical sensor configured to detect changes of reflectivity of a chromogenic film at a bottom of the sample container, wherein the chromogenic film is configured to generate a chromogenic reaction with acidic substances produced during culture, and the detection curve, the metabolism curve, and the growth curve are curves representing changes of the reflectivity over culture time; or
wherein the detection sensor is a fluorescence sensor configured to detect changes of fluorescence intensity of a fluorescent substance in the sample container, the fluorescence intensity changes with the growth of the micro-organisms, and the detection curve, the metabolic curve, and the growth curves are curves representing changes of the fluorescence intensity over culture time.

13. The blood culture detection system of claim 1, wherein the first information is a detection curve generated based on detection signals of the blood sample at different culture times; the controller configured to: if the first information meets a first preset condition within the first preset time period, label and output a detection status of the blood sample as “positive”, comprises: the controller configured to:
if the detection curve meets the first preset condition within the first preset time period, label and output the detection status of the blood sample as “positive”,
wherein the first preset condition comprises at least one of:
there continuously occurs a fourth number of numerical values of the detection curve that are equal to or greater than a fourth threshold, or a time interval in which numerical values of the detection curve are continuously equal to or greater than a fourth threshold is equal to or greater than a fourth time interval threshold;
there continuously occurs a fifth number of first-order derivatives of the detection curve that are equal to or greater than a fifth threshold, or a time interval in which first-order derivatives of the detection curve are continuously equal to or greater than a fifth threshold is equal to or greater than a fifth time interval threshold;
there continuously occurs a sixth number of second-order derivatives of the detection curve that are equal to or greater than a sixth threshold, or a time interval in which second-order derivatives of the detection curve are continuously equal to or greater than a sixth threshold is equal to or greater than a sixth time interval threshold; or
both first-order derivatives and second-order derivatives of the detection curve are equal to or greater than their corresponding preset thresholds, either for a time interval that is equal to or greater than a preset time interval, or for a number of data points that is equal to or greater than a preset number.

14. The blood culture detection system of claim 1, wherein the controller is further configured to:
during culture, continuously perform judgment on the first information based on the first preset condition and continuously perform judgment on the third information based on the second preset condition; and if a culture time reaches an end time of the first preset time period and the detection status of the blood sample has not been labeled as “positive”, send a prompt of extending culture to a user or automatically extend culture; or
during culture, continuously perform judgment on the first information based on the first preset condition and continuously perform judgment on the third information based on the second preset condition; if the first information does not meet the first preset condition and the third information does not meet the second preset condition, label and output the detection status of the blood sample as “negative so far”; and if a culture time reaches an end time of the first preset time period and the detection status of the blood sample is still labeled as “negative so far”, send a prompt of extending culture to a user or automatically extend culture; or
during culture, continuously perform judgment on the first information based on the first preset condition and continuously perform judgment on the third information based on the second preset condition; and if a culture time reaches an end time of the first preset time period and the detection status of the blood sample has not been labeled as “positive”, label and output the detection status of the blood sample as “negative”; or
during culture, continuously perform judgment on the first information based on the first preset condition and continuously perform judgment on the third information based on the second preset condition; if the first information does not meet the first preset condition and the third information does not meet the second preset condition, label and output the detection status of the blood sample as “negative so far”; and if a culture time reaches an end time of the first preset time period and the detection status of the blood sample is still labeled as “negative so far”, label and output the detection status of the blood sample as “negative”.

15. A blood culture detection system, comprising:
an incubation module, comprising an incubation component and a temperature control component, wherein the incubation component is configured to hold at least one sample container carrying a blood sample and a culture substrate and to culture micro-organisms within the blood sample at a preset temperature, and the temperature control component is configured to regulate the preset temperature;
a detection module, comprising at least one detection sensor configured to detect the blood sample and to generate a detection signal of the blood sample; and
a controller, configured to:
acquire and process the detection signal of the blood sample, so as to obtain first information of the blood sample;
acquire second information related to metabolism of blood cells within the blood sample in the culture substrate of the sample container; and
obtain third information related to growth of the micro-organisms within the blood sample in the culture substrate of the sample container, based on the first information and the second information.

16. The blood culture detection system of claim 15, wherein the first information is a detection curve generated based on detection signals of the blood sample at different culture times, the second information is a metabolism curve related to metabolism of the blood cells within the blood sample in the culture substrate of the sample container at different culture times, and the third information is a growth curve related to growth of the micro-organisms within the blood sample in the culture substrate of the sample container at different culture times; the controller configured to: obtain third information related to growth of the micro-organisms within the blood sample in the culture substrate of the sample container, based on the first information and the second information, comprises the controller configured to:
process the detection curve according to the metabolism curve, so as to obtain the growth curve; and/or
subtract the metabolism curve from the detection curve, so as to obtain the growth curve.

17. The blood culture detection system of claim 15, wherein the controller is further configured to:
analyze the third information, so as to obtain an analysis result, and
label and output a detection status of the blood sample as “positive” based on the analysis result, or label and output the detection status of the blood sample as “negative so far” based on the analysis result, or label and output the detection status of the blood sample as “negative” based on the analysis result, or send a prompt of extending culture to a user based on the analysis result, or automatically extend culture of the blood sample based on the analysis result;
and/or,
wherein the blood culture detection system further comprises a display, and the controller is further configured to control the display to display the third information.

18. A sample detection method, comprising:
acquiring, by a detection module, a detection signal of a blood sample, and processing the detection signal to obtain first information of the blood sample;
acquiring second information related to metabolism of blood cells within the blood sample in a culture substrate of a sample container; and
obtaining third information related to growth of micro-organisms within the blood sample in the culture substrate of the sample container, based on the first information and the second information.

19. The sample detection method of claim 18, wherein the first information is a detection curve generated based on detection signals of the blood sample at different culture times, the second information is a metabolism curve related to metabolism of the blood cells within the blood sample in the culture substrate of the sample container at different culture times, and the third information is a growth curve related to growth of the micro-organisms within the blood sample in the culture substrate of the sample container at different culture times; obtaining third information related to growth of the micro-organisms within the blood sample in the culture substrate of the sample container, based on the first information and the second information comprises:
processing the detection curve according to the metabolism curve, to obtain the growth curve; and/or
subtracting the metabolism curve from the detection curve, to obtain the growth curve.

20. The sample detection method of claim 18, further comprising:
analyzing the third information to obtain an analysis result, and
labeling and outputting a detection status of the blood sample as “positive” based on the analysis result, or labeling and outputting the detection status of the blood sample as “negative so far” based on the analysis result, or labeling and outputting the detection status of the blood sample as “negative” based on the analysis result, or sending a prompt of extending culture to a user based on the analysis result, or automatically extending culture of the blood sample based on the analysis result;
and/or,
controlling a display to display the third information.
